(19) United States
(12) Patent Application Publication
(10) Pub. No.: US 2026/0284217 A1
(43) Pub. Date: Sep. 24, 2026

(54) Enzymatic Removable Cell Penetration-Blocker (rCPB) Conjugated To A Tandem Peptide-Cytotoxin, Tandem Peptide-miRNA Chimeric Molecules Or Tandem Peptide-Antioncogenic miRNA For Targeted Cancer Cell Delivery, Off-Target Toxicity Minimization, Resistance Reduction, Gene Regulation, Anti-Tumor Immune Response Upregulation In The Tumor Microenvironment And Systemically, And Synergistic Enhancement Of Anti-Tumor Activity

(71) Applicant: Xiangfei Cheng, Montgomery Village, MD (US)

(72) Inventor: Xiangfei Cheng, Montgomery Village, MD (US)

(21) Appl. No.: 19/088,716

(22) Filed: Mar. 24, 2025

(51) Int. Cl. A61K 47/68; C07K 16/28

(52) U.S. Cl. CPC A61K 47/6829; A61K 47/6807; A61K 47/6849; C07K 16/2827

(57) ABSTRACT

My present innovative rCPB-conjugated tandem peptide-RNA, rCPB-conjugated NoCltandem peptide-RNA, rCPB-conjugated tandem peptide-cytotoxin and rCPB-conjugated NoCltandem peptide-cytotoxin are designed with a three-layered mechanism to minimize off-target toxicity: (1) targeting peptide directs conjugate or chimeric molecule to target cells; (2) rCPB is removed through breaking N-(-glutamyl) lysine isopeptide bond by TG highly expressed on cancers cells to enable rCPB-free conjugate or rCPB-free chimeric molecule to deliver into cancer cells, thereby ensuring that normal cells are minimally affected, even within the same tissue; and (3) active components (anti-PD-L1, cytotoxin, miRNA, antioncogenic miRNA) is released through cleaving specifically designed linkers by cathepsin B highly expressed in cancer cells to exert synergistic roles in immune response and gene regulation or directly kill cancer cells upon delivery into cancer cells. Taken together, the molecules achieve targeted delivery to cancer cells highly expressing TG, while sparing normal cells coexisting in the same tissue, thereby reducing off-target toxicity.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A SEQUENCE LISTING, A LARGE TABLE, OR A COMPUTER PROGRAM LISTING APPENDIX ON READ-ONLY OPTICAL DISC

The present application includes a sequence Listing in XML format, which was submitted as a separate file via the USPTO's EFS-Web system. The Sequence Listing XML, with the file name, and Peptide_Sequence_19088716_Xiangfei.xml, is incorporated by reference in its entirety as part of this disclosure. The sequence Listing contains peptide sequences for the following peptides: RGD4C (SEQ ID NO: 1), iRGD1 (SEQ ID NO: 2), iRGD2 (SEQ ID NO: 3), LyP-1 (SEQ ID NO: 4), K237 (SEQ ID NO: 5), mUNO (SEQ ID NO: 6), Octreotide (SEQ ID NO: 7), IL4RPep-1 (SEQ ID NO: 8), EPI-X4 (SEQ ID NO: 9), TV1 (SEQ ID NO: 10), Bld-1 (SEQ ID NO: 11), AAD (SEQ ID NO: 12), GP-5 (SEQ ID NO: 13), LN1 (SEQ ID NO: 14), H3 (SEQ ID NO: 15), Prost1 (SEQ ID NO: 16), GE11 (SEQ ID NO: 17), GIRLRG peptide (SEQ ID NO: 18), hspb1 (SEQ ID NO: 19), OSP-1 (SEQ ID NO: 20), nucleolin-binding F3 peptide (SEQ ID NO: 21), BIRD2 (SEQ ID NO: 22), S7A (SEQ ID NO: 23), R11A (SEQ ID NO: 24), transportan (SEQ ID NO: 25), anti-PD-L1 peptide (SEQ ID NO: 26), first linker (SEQ ID NO: 27), second linker (SEQ ID NO: 28), third linker (SEQ ID NO: 29) and fourth linker (SEQ ID NO: 30). The file is incorporated by reference in its entirety. The sequence Listing is provided in a separate document (Sequence Listing file) in compliance with the requirements for submitting sequence data in the appropriate format. The XML file contains the complete peptide sequences as defined by the listed peptides, and is hereby incorporated by reference as part of the application. The XML file information and the following sequences are provided in the accompanying Sequence Listing and are referenced throughout this application by their respective SEQ ID numbers. The Sequence Listing is incorporated by reference in accordance with 37 CFR 1.835 and includes the following details:
1) Name of XML File: Peptide_Sequence_19088716_Xiangfei.xml
2) Date of Creation: Jun. 17, 2026
3) Size of XML File: 37.3 KB

The SEQ ID numbers (SEQ ID NO) assigned in the Sequence Listing correspond to the following peptide sequences, as represented in the accompanying XML file:
SEQ ID NO: 1 (sequenceIDNumber=“1”)-RGD4C;
SEQ ID NO: 2 (sequenceIDNumber=“2”)-iRGD1;
SEQ ID NO: 3 (sequenceIDNumber=“3”)-iRGD2;
SEQ ID NO: 4 (sequenceIDNumber=“4”)-LyP-1;
SEQ ID NO: 5 (sequenceIDNumber=“5”)-K237;
SEQ ID NO: 6 (sequenceIDNumber=“6”)-mUNO;
SEQ ID NO: 7 (sequenceIDNumber=“7”)-Octreotide;
SEQ ID NO: 8 (sequenceIDNumber=“8”)-IL4RPep-1;
SEQ ID NO: 9 (sequenceIDNumber=“9”)-EPI-X4;
SEQ ID NO: 10 (sequenceIDNumber=“10”)-TV1;
SEQ ID NO: 11 (sequenceIDNumber=“11”)-Bld-1;
SEQ ID NO: 12 (sequenceIDNumber=“12”)-AAD;
SEQ ID NO: 13 (sequenceIDNumber=“13”)-GP-5;
SEQ ID NO: 14 (sequenceIDNumber=“14”)-LN1;
SEQ ID NO: 15 (sequenceIDNumber=“15”)-H3;
SEQ ID NO: 16 (sequenceIDNumber=“16”)-Prost1;
SEQ ID NO: 17 (sequenceIDNumber=“17”)-GE11;
SEQ ID NO: 18 (sequenceIDNumber=“18”)-GIRLRG peptide;
SEQ ID NO: 19 (sequenceIDNumber=“19”)-hspb1;
SEQ ID NO: 20 (sequenceIDNumber=“20”)-OSP-1;
SEQ ID NO: 21 (sequenceIDNumber=“21”)-Nucleolin-binding F3 peptide;
SEQ ID NO: 22 (sequenceIDNumber=“22”)-BIRD2;
SEQ ID NO: 23 (sequenceIDNumber=“23”)-S7A;
SEQ ID NO: 24 (sequenceIDNumber=“24”)-R11A;
SEQ ID NO: 25 (sequenceIDNumber=“25”)-Transportan;
SEQ ID NO: 26 (sequenceIDNumber=“26”)-Anti-PD-L1 Peptide;
SEQ ID NO: 27 (sequenceIDNumber=“27”)-First Linker;
SEQ ID NO: 28 (sequenceIDNumber=“28”)-Second Linker;
SEQ ID NO: 29 (sequenceIDNumber=“29”)-Third Linker;
SEQ ID NO: 30 (sequenceIDNumber=“30”)-Fourth Linker;

Each peptide sequence is described in detail within the Sequence Listing, and their use in the invention is outlined in the relevant sections of this specification. RGD is a peptide of fewer than four amino acids. It is not included in the Sequence Listing XML, in accordance with ST.26.

### BACKGROUND OF THE INVENTION

Cancer therapy is often limited by the development of off-target toxicity. In my present invention, I designed enzymatic removable cell penetration blocker (rCPB) and a linker, CQGG (SEQ ID NO:30), to construct conjugate and chimeric molecules for intervention in cancers highly expressing members of transglutaminase (TG) family. Transglutaminases are enzymes that in nature primarily catalyze the formation of an N(-glutamyl) lysine isopeptides (γ:ε linkage) between γ-carboxamide groups (—(C═O)NH2) of glutamine residue side chains and the ¿-amino groups (—NH2) of lysine residue side chains with subsequent release of ammonia (NH3). The transglutaminases play a dynamic role in biology not only by promoting the formation but also the breaking of N(-glutamyl) lysine isopeptides (γ:ε linkage) (1). In my present innovation, all members of the TG family are referred to as TG.

My earlier patent applications, U.S. patent application Ser. No. 19/074,994 and U.S. patent application Ser. No. 19/017,738, introduced foundational platforms for conjugates and chimeric molecules aimed at targeted delivery for interventions in cancers. While these technologies laid important groundwork, my present innovation provides a novel mechanism for precise targeted delivery and significant reduction in off-target toxicity. In my present innovation, I have invented rCPB-Conjugated tandem peptide-cytotoxin, rCPB-Conjugated tandem peptide-miRNA chimeric molecules, rCPB-Conjugated tandem peptide-antioncogenic miRNA chimeric molecules, rCPB-conjugated NoCltandem peptide-miRNA chimeric molecule, rCPB-conjugated NoCltandem peptide-antioncogenic miRNA chimeric molecule, and rCPB-conjugated NoCltandem peptide-cytotoxin to enhance precision delivery and reduce off-target toxicity by introducing rCPB and a linker CQGG (SEQ ID NO: 30) into the strategy outlined in my earlier patent applications, U.S. patent application Ser. No. 19/074,994 and U.S. patent application Ser. No. 19/017,738. In the present innovative molecules, the conjugate or chimeric molecule can't deliver into cancer cells without rCPB removal through cleavage of N-(-glutamyl) lysine isopeptide bond by TG expressed on cancer cells. Therefore, these innovative molecules are specifically for intervention in cancers highly expressing TG. The role of linker CQGG (SEQ ID NO: 30), rCPB and N-(-glutamyl) lysine isopeptide bond will be described in more detail in the following sections. This advancement specifically targets cancers that highly express TG which can specifically remove rCPB, enabling the precisely targeted delivery of the rCPB-free conjugate or rCPB-free chimeric molecule into the cancers highly expressing TG, to reduce off-target toxicity and enhance efficacy. This is distinct from my earlier patent applications, U.S. patent application Ser. No. 19/074,994 and U.S. patent application Ser. No. 19/017,738. Notably, my present patent application is a standalone filing and does not claim priority to, nor is it a continuation of, any previously filed applications (application Ser. No. 19/074,994 and application Ser. No. 19/017,738). My earlier two applications (application Ser. No. 19/074,994 and application Ser. No. 19/017,738) are configured for intervention in a broader range of cancers. The present innovations are specifically configured for intervention in cancers with high expression of TG.

As used in the present invention, a component conjugated to a cytotoxin is referred to as a ‘conjugate,’ whereas a component conjugated to RNA (miRNA or antioncogenic miRNA) is referred to as a ‘chimeric molecule’.

The tumor microenvironment (TME) is a key factor influencing the response to immunotherapy. Cold tumors—characterized by a lack of immune cell infiltration and an immunosuppressive environment—are often resistant to immunotherapies. Tumor-infiltrating immune cells can have both pro-tumor and anti-tumor effects, greatly affecting tumor progression and the success of cancer treatments. Converting a cold tumor environment into a “hot” one is a major challenge but is essential for enhancing the efficacy of immunotherapies.

Despite recent advancements, there remains an urgent need for novel therapeutic strategies that simultaneously overcome drug resistance, off-target toxicity and target downregulation. TG is highly expressed in pancreatic tumor, breast cancer, ovarian cancer, epidermal squamous cell carcinoma, prostate cancer, melanoma, lung cancer, colon cancer, acute myeloid leukemia (AML), bladder cancer, hepatocarcinoma cancer, gastric cancer, renal cell carcinoma, cervical cancer, mantle cell lymphoma, osteosarcoma, esophageal cancer, mesothelioma and laryngeal cancer, with TG2 being the predominant subtype. In my present innovation, I have innovated molecules specifically targeting cancers which highly express TG to reduce off-target toxicity, to enhance efficacy and decrease resistance development through targeting multiple pathways, including gene regulation, enhancing anti-tumor immune response, converting a cold tumor environment into a “hot” one and directly killing cancer cells by cytotoxin.

### BRIEF SUMMARY OF THE INVENTION

TG is a family of structurally and functionally related proteins that catalyze the Ca²⁺-dependent posttranslational modification of proteins by introducing covalent bonds between free amine groups (e.g., protein- or peptide-bound lysine) and γ-carboxamide groups of peptide-bound glutamines. Transglutaminase not only catalyze N-(-glutamyl) lysine cross-links, connecting various peptide chain segments but also hydrolyze the bond (γ:ε linkage) (1). TG is highly expressed in various tumors (cancers), including pancreatic cancer, breast cancer, ovarian cancer, epidermal squamous cell carcinoma, prostate cancer, glioma, melanoma, lung cancer, colon cancer, AML, bladder cancer, Hepatocarcinoma cancer, Gastric Cancer, Renal cell carcinoma, Cervical cancer, Mantle cell lymphoma, Osteosarcoma, Esophageal cancer, Mesothelioma, Laryngeal Cancer and thyroid cancers (2, 3). TG is localized in the cell membrane and cytoplasm (4). In my present invention, I have designed rCPB and fourth linker, CQGG (SEQ ID NO: 30), for forming N-(-glutamyl) lysine isopeptide bond, specifically cleavable by TG expressed on cancer cells, to develop a novel mechanism targeting cancers highly expressing TG. By leveraging this mechanism, I have developed innovative molecules specifically designed for intervention in cancers characterized by high expression of the TG. I have integrated targeted delivery, precisely recognizing cancer cells through multiple layers of recognition mechanisms, cytotoxic and pro-immune response functions, all within a single molecule, the rCPB-conjugated tandem peptide-cytotoxin. This approach also promotes the conversion of a cold tumor microenvironment into a hot one, while reducing resistance development.

rCPB-conjugated tandem peptide-Cytotoxin includes rCPB-conjugated tandem peptide-DOX and rCPB-conjugated tandem peptide-LLO. The innovative conjugates are created by conjugating tandem peptide to DOX or LLO at C-terminus of tandem peptide, subsequently conjugating rCPB to tandem peptide-DOX or tandem peptide-LLO through the linker, CQGG (SEQ ID NO: 30), which is conjugated to the rCPB. These innovative conjugates integrate the targeted delivery, reducing off-target toxicity and resistance development by dual-binding to target cells, cleavage of linkers specifically by cathepsin B expressed in cancer cells and N-(-glutamyl) lysine isopeptide bond by TG expressed on cancer cells, respectively, within a single molecule. Both TG and cathepsin B are highly expressed in cancer cells. By integrating their individual roles within a signal molecule, the innovative molecule synergistically enhances cancer cell eradication through inhibiting PD-L1 by a component of the conjugate, anti-PD-L1 peptide (SEQ ID NO: 26), converting a cold tumor microenvironment into a hot one, and directly inducing cancer cell death by toxic agents, DOX or LLO, leading to enhance efficacy, reduce the development of resistance, minimize off-target toxicity, and decrease dose. In my present innovation description, DOX and LLO are referred to as cytotoxic agents or cytotoxin.

Additionally, I have innovated the rCPB-conjugated tandem peptide-RNA chimeric molecule which integrates targeted delivery through precise cancer cell recognition and gene regulation within a single molecule. The rCPB-conjugated tandem peptide-RNA chimeric molecule includes rCPB-conjugated tandem peptide-miRNA chimeric molecules and rCPB-conjugated tandem peptide-antioncogenic miRNA chimeric molecules. The innovative chimeric molecules are created by conjugating tandem peptide to 3′ end of passenger strand of double-stranded miRNA or 3′ end of passenger strand of double-stranded antioncogenic miRNA through the third linker, GRRC (SEQ ID NO: 29), subsequently conjugating rCPB to the tandem peptide-miRNA chimeric molecule or the tandem peptide-antioncogenic miRNA chimeric molecule through the fourth linker, CQGG (SEQ ID NO: 30), which is conjugated to rCPB. These innovative conjugates integrate the targeted delivery, reducing off-target toxicity and resistance development by dual-binding to target cells, cleavage of the designed linkers specifically by cathepsin B expressed in cancer cells and N-(-glutamyl) lysine isopeptide bond by TG expressed on cancer cells, respectively. By integrating their individual roles within a signal molecule, the innovative molecules are designed to precisely targeted delivery and synergistically enhance cancer cell eradication through gene regulation and upregulation of anti-tumor immune response at tumor microenvironment and systematically, leading to enhance efficacy, reduce the development of resistance, minimize off-target toxicity, and decrease dose.

In present innovation, I have furthermore innovated rCPB-conjugated NoCltandem peptide-RNA chimeric molecule, which includes rCPB-conjugated NoCltandem peptide-miRNA chimeric molecules and rCPB-conjugated NoCltandem peptide-antioncogenic miRNA chimeric molecules. NoCltandem peptide refer to the tandem peptide that does not contain anti-PD-L1 peptide (SEQ ID NO: 26).

In present innovation, I have also innovated rCPB-conjugated NoCltandem peptide-cytotoxin which includes rCPB-conjugated NoCltandem peptide-DOX and rCPB-conjugated NoCltandem peptide-LLO.

Importantly, in my present innovations, conjugates and chimeric molecules can't be delivered into cancer cells without removing rCPB from the conjugates and the chimeric molecules through cleavage of N-(γ-glutamyl) lysine isopeptide bond by TG expressed on cancer cells. This approach significantly reduces off-target toxicity. This is significantly distinct from my earlier patent applications, U.S. patent application Ser. No. 19/074,994 and U.S. patent application Ser. No. 19/017,738. Notably again, this application is a standalone filing and does not claim priority to, nor is it a continuation of, any previously filed applications. A detailed description of N-(γ-glutamyl) lysine isopeptide bond is provided in the following section.

While the individual components of the conjugate and chimeric molecule—such as transportan (SEQ ID NO: 25), targeting peptide, cytotoxin, miRNA, antioncogenic miRNA and anti-PD-L1 peptide (SEQ ID NO: 26)—are known in the art, their novel combination and integration into a single molecular entity that addresses multiple challenges in cancer therapy (such as targeting, off-target toxicity, immune activation, and resistance) has not been previously disclosed or suggested.

The present invention strategically combines these elements in a unique configuration, utilizing a cleavable linker to selectively release active components, while exerting synergistic roles by simultaneously regulating gene expression within the tumor microenvironment and activating immune responses both locally within the TME and systemically. This combination results in a synergistic effect, including enhanced targeting, reduced off-target toxicity, and minimized resistance development, none of which are achieved in existing approaches.

In particular, in these innovative molecules, I have designed enzymatic removable cell penetration blocker (rCPB) and fourth linker, CQGG (SEQ ID NO: 30), for conjugating rCPB to tandem peptide or to NoCltandem peptide by forming N-(γ-glutamyl) lysine isopeptide bond between glutamine (Q) in fourth linker, CQGG (SEQ ID NO: 30), and lysine in tandem peptide or between glutamine (Q) in fourth linker, CQGG (SEQ ID NO: 30), and lysine in NoCltandem peptide. The N-(γ-glutamyl) lysine isopeptide bond (γ:ε linkage) is breakable by TG highly expressed on cancer cell for targeted delivery within this multifunctional construct. The tandem peptide-cytotoxin, tandem peptide-RNA chimeric molecule, NoCltandem peptide-RNA chimeric molecule and NoCltandem peptide-cytotoxin can't be delivered into cancer cells without removing rCPB through breaking N-(γ-glutamyl) lysine isopeptide bond formed between glutamine (Q) in fourth linker, CQGG (SEQ ID NO: 30), and lysine in tandem peptide or between glutamine (Q) in fourth linker, CQGG (SEQ ID NO: 30), and lysine in NoCltandem peptide by TG highly expressed on cancer cells to achieve targeted delivery and minimize off-target toxicity.

In the present invention, the bond formed between glutamine (Q) in fourth linker, CQGG (SEQ ID NO: 30), conjugated to rCPB and lysine (K) in the tandem peptide, or alternatively between glutamine (Q) in fourth linker, CQGG (SEQ ID NO: 30), conjugated to rCPB and lysine (K) in the NoCltandem peptide, is referred to as the N-(γ-glutamyl) lysine isopeptide bond.

My present innovative molecules are designed with a three-layered mechanism to minimize off-target toxicity: (1) targeting peptide in NoCltandem peptide or both targeting peptide and anti-PD-L1 peptide (SEQ ID NO: 26) in tandem peptide directs conjugate or chimeric molecule to target cells by binding to their targets; (2) rCPB is removed through breaking N-(γ-glutamyl) lysine isopeptide bond by TG highly expressed on cancers cells to enable rCPB-free conjugate or rCPB-free chimeric molecule to deliver into the cancer cells, thereby ensuring that normal cells are minimally affected, even within the same tissue; and (3) active components (such as anti-PD-L1, cytotoxin, miRNA, antioncogenic miRNA) is released through cleavage of first, second and third linkers by cathepsin B highly expressed in cancers in cytoplasm to exert synergistic roles in killing cancer cells. Taken together, the molecules achieve targeted delivery specifically to cancer cells highly expressing TG, while sparing normal cells that coexist in the same tissue, thereby reducing off-target toxicity.

Moreover, the use of this multi-functional design to achieve such effects is not obvious to a person skilled in the art, as no prior art suggests combining these components in the manner described, nor would a skilled person have a reason to do so without the inventive insight provided by this disclosure.

### BRIEF DESCRIPTION OF THE SEVERAL VIEWS OF THE DRAWING

FIG. 1: Structure of rCPB-conjugated Tandem Peptide-Cytotoxin, DOX or LLO. The tandem peptide comprises transportan (SEQ ID NO: 25), first linker KGRRG (SEQ ID NO: 27), targeting peptide, second linker GRRG (SEQ ID NO: 28), anti-PD-L1 peptide (SEQ ID NO: 26) and third linker GRRC (SEQ ID NO: 29) from N-terminus to C-terminus. Tandem peptide is conjugated to DOX or LLO through the linker at C-terminus of tandem peptide. Fourth linker is CQGG (SEQ ID NO: 30). rCPB is conjugated to tandem peptide by forming N-(-glutamyl) lysine isopeptide bond between glutamine (Q) in CQGG (SEQ ID NO: 30) and lysine (K) in tandem peptide catalyzed by TG. The targeting peptide is selected based on its binding specificity to different target cells. The first, second and third linkers are specifically cleavable by cathepsin B which is highly expressed in cancer cells compared to normal cells. The N-(γ-glutamyl) lysine isopeptide bond between glutamine (Q) in CQGG (SEQ ID NO: 30) and lysine (K) in tandem peptide is breakable by TG highly expressed on cancer cells.

FIG. 2: Structure of rCPB-conjugated Tandem Peptide-miRNA Chimeric Molecule and rCPB-conjugated Tandem Peptide-antioncogenic miRNA Chimeric Molecule. The tandem peptide comprises transportan (SEQ ID NO: 25), first linker KGRRG (SEQ ID NO: 27), targeting peptide, second linker GRRG (SEQ ID NO: 28), anti-PD-L1 peptide (SEQ ID NO: 26) and third linker GRRC (SEQ ID NO: 29) from N-terminus to C-terminus. Tandem peptide is conjugated to 3′ end of passenger strand of miRNA or 3′ end of passenger strand of antioncogenic miRNA through third linker. Fourth linker is CQGG (SEQ ID NO: 30). rCPB is conjugated to tandem peptide by forming N-(-glutamyl) lysine isopeptide bond between glutamine (Q) in CQGG (SEQ ID NO: 30) and lysine (K) in tandem peptide catalyzed by TG. The targeting peptide is selected based on its binding specificity to different target cells. The first, second and third linkers are specifically cleavable by cathepsin B which is highly expressed in cancer cells compared to normal cells. The N-(γ-glutamyl) lysine isopeptide bond between glutamine (Q) in CQGG (SEQ ID NO: 30) and lysine (K) in tandem peptide is breakable by TG highly expressed on cancer cells. miRNA and antioncogenic miRNA are double strands.

FIG. 3: Structure of rCPB-conjugated NoCltandem Peptide-miRNA Chimeric Molecule and rCPB-conjugated NoCltandem Peptide-antioncogenic miRNA Chimeric Molecule. The tandem peptide comprises transportan (SEQ ID NO: 25), first linker KGRRG (SEQ ID NO: 27), targeting peptide, and third linker GRRC (SEQ ID NO: 29) from N-terminus to C-terminus. NoCltandem peptide is conjugated to 3′ end of passenger strand of miRNA or 3′ end of passenger strand of antioncogenic miRNA through third linker. Fourth linker is CQGG (SEQ ID NO: 30). rCPB is conjugated to NoCltandem peptide by forming N-(-glutamyl) lysine isopeptide bond between glutamine (Q) in CQGG (SEQ ID NO: 30) and lysine (K) in NoCltandem peptide catalyzed by TG. The targeting peptide is selected based on its binding specificity to different target cells. The first and third linkers are specifically cleavable by cathepsin B which is highly expressed in cancer cells compared to normal cells. The N-(γ-glutamyl) lysine isopeptide bond between glutamine (Q) in fourth linker CQGG (SEQ ID NO: 30) and lysine (K) in NoCltandem peptide is breakable by TG highly expressed on cancer cells. miRNA and antioncogenic miRNA are double strands. rCPB-conjugated tandem peptide-miRNA chimeric molecule and rCPB-conjugated tandem peptide-antioncogenic miRNA chimeric molecule contain the first, second, third, and fourth linkers, while the rCPB-conjugated NoCltandem peptide-miRNA chimeric molecule and rCPB-conjugated NoCltandem peptide-antioncogenic miRNA chimeric molecule lack the second linker. To maintain consistency, the linker names (first, third, and fourth) are kept the same in the rCPB-conjugated NoCltandem peptide-miRNA chimeric molecule and rCPB-conjugated NoCltandem peptide-antioncogenic miRNA chimeric molecule, even though the second linker is absent.

FIG. 4: Structure of rCPB-conjugated NoCltandem Peptide-DOX and rCPB-conjugated NoCltandem Peptide-LLO. The tandem peptide comprises transportan (SEQ ID NO: 25), first linker KGRRG (SEQ ID NO: 27), targeting peptide, and third linker GRRC (SEQ ID NO: 29) from N-terminus to C-terminus. NoCltandem peptide is conjugated to DOX or LLO through third linker at C-terminus of NoCltandem peptide. Fourth linker is CQGG (SEQ ID NO: 30). rCPB is conjugated to NoCltandem peptide by forming N-(-glutamyl) lysine cross-links between glutamine (Q) in CQGG (SEQ ID NO: 30) and lysine (K) in NoCltandem peptide catalyzed by TG. The targeting peptide is selected based on its binding specificity to different target cells. The first and third linkers are specifically cleavable by cathepsin B which is highly expressed in cancer cells compared to normal cells. The N-(γ-glutamyl) lysine isopeptide bond between glutamine (Q) in fourth linker CQGG (SEQ ID NO: 30) and lysine (K) in NoCltandem peptide is breakable by TG highly expressed on cancer cells. rCPB-conjugated tandem peptide-cytotoxin contain the first, second, third, and fourth linkers, while the rCPB-conjugated NoCltandem peptide-DOX and rCPB-conjugated NoCltandem peptide-LLO lack the second linker. To maintain consistency, the linker names (first, third, and fourth) are kept the same in the rCPB-conjugated NoCltandem peptide-DOX and rCPB-conjugated NoCltandem peptide-LLO, even though the second linker is absent.

FIG. 5. The Targeted Delivery and Mechanism of Action of rCPB-conjugated Tandem Peptide-DOX. a) rCPB-conjugated Tandem Peptide-DOX binds to cancer cells through specific dual-binding of tandem peptide to cancer cells in which targeting peptide specifically binds to target cell and anti-PD-L1 peptide (SEQ ID NO: 26) specifically binds to PD-L1, while rCPB is removed from the conjugate through cleavage of N-(-glutamyl) lysine isopeptide bond by TG highly expressed on cancer cells. Upon removing rCPB, tandem Peptide-Conjugated DOX is specifically delivered into cancer cells through synergistic role with the intracellular trafficking peptide, transportan (SEQ ID NO: 25), a component of the conjugate, in crossing cell membrane; b) Following the targeted delivery of the rCPB-free conjugate into cancer cells, the first, second and third linkers in the conjugate are cleaved by cathepsin B which is highly expressed in cancer cells, leading to release of anti-PD-L1 peptide (SEQ ID NO: 26), DOX and targeting peptide in cytoplasm; c) Anti-PD-L1 peptide (SEQ ID NO: 26) inhibit PD-L1. DOX damages DNA in nucleus, leading to cancer cell apoptosis; d). PD-1 and PD-L1 interaction is blocked by anti-PD-L1 peptide (SEQ ID NO: 26), leading to attenuating T cell exhaustion, thereby enhances anti-tumor immune response both in tumor microenvironment and systemic immune response, and DOX induces immunogenic cell death, which promotes high DAMP signals for DC maturation and cytotoxic T lymphocyte activation, resulting in an increased anti-tumor immune response in the tumor microenvironment and a heightened systemic immune response; e) Upregulate the anti-tumor immune response via blockage of PD-1 and PD-L1 interaction by anti-PD-L1 peptide (SEQ ID NO: 26), cytokines released by immune cells and signals from the tumor microenvironment; f) Immune cells infiltrate tumors, leading to converting a cold tumor microenvironment into a hot one and eliminating tumor.

FIG. 6. The Targeted Delivery and Mechanism of Action of The rCPB-conjugated Tandem Peptide-LLO. a) rCPB-conjugated Tandem Peptide-LLO binds to cancer cells through specific dual-binding of tandem peptide to cancer cells in which targeting peptide specifically binds to target cell and anti-PD-L1 peptide (SEQ ID NO: 26) specifically binds to PD-L1, while rCPB is removed from the conjugate via cleavage of N-(-glutamyl) lysine isopeptide bond by TG highly expressed on cancer cells. Upon removing rCPB, tandem Peptide-LLO is specifically delivered into cancer cells through synergistic role with the intracellular trafficking peptide, transportan (SEQ ID NO: 25), a component of the conjugate, in crossing cell membrane; b) Following the targeted delivery of the rCPB-free conjugate into cancer cells, the first, second and third linkers in the conjugate are cleaved by cathepsin B which is highly expressed in cancer cells, leading to release of anti-PD-L1 peptide (SEQ ID NO: 26), LLO and targeting peptide in cytoplasm; c) Anti-PD-L1 peptide (SEQ ID NO: 26) inhibit PD-L1. LLO disrupts the phagosomal membrane; d) PD-1 and PD-L1 interaction is blocked by anti-PD-L1 peptide (SEQ ID NO: 26), leading to attenuating T cell exhaustion, thereby enhances the anti-tumor immune response both in the tumor microenvironment and systemically. The LLO disrupts phagolysosomal membranes that enhance antigen cross-presentation of tumor-derived peptides and activate cytosolic immune sensors, and boost macrophage activation to a pro-inflammatory phenotype, leading to enhancing anti-tumor immune response both in tumor microenvironment and systemic immune response; e) Upregulate the anti-tumor immune response via blockage of PD-1 and PD-L1 interaction by anti-PD-L1 peptide (SEQ ID NO: 26), cytokines released by immune cells and signals from the tumor microenvironment; f) Immune cells infiltrate tumors, leading to converting a cold tumor microenvironment into a hot one and eliminating tumor.

FIG. 7. The Targeted Delivery and Mechanism of Action of The rCPB-conjugated Tandem Peptide-miRNA Chimeric Molecule. a) rCPB-conjugated Tandem Peptide-miRNA Chimeric Molecule binds to cancer cells through specific dual-binding of tandem peptide to cancer cells in which targeting peptide specifically binds to target cell and anti-PD-L1 peptide (SEQ ID NO: 26) specifically binds to PD-L1, while rCPB is removed from the chimeric molecule via cleavage of N-(-glutamyl) lysine isopeptide bond by TG highly expressed on cancer cells. Upon removing rCPB, the tandem peptide-miRNA chimeric molecule is specifically delivered into cancer cells through synergistic role with the intracellular trafficking peptide, transportan (SEQ ID NO: 25), a component of the conjugate, in crossing cell membrane; b) Following the targeted delivery of the tandem peptide-miRNA chimeric molecule into cancer cells, the double strands of miRNA dissociate, allowing the guide strand to be selected for loading into Argonaute, forming RISC, and then bind to target mRNA while the first, second and third linkers are cleaved by cathepsin B which is highly expressed in cancer cells, leading to release of anti-PD-L1 peptide (SEQ ID NO: 26), passenger strand of miRNA and targeting peptide in cytoplasm; c) Anti-PD-L1 peptide (SEQ ID NO: 26) inhibit PD-L1. Binding of guide strand of miRNA to mRNA within RISC leads translational repression or the targeted mRNA degradation, while the passenger strands are ejected and degraded; (d) Binding of miRNA to mRNA results in cancer cells apoptosis by translational repression or inducing the target mRNA degradation; PD-1 and PD-L1 interaction is blocked by anti-PD-L1 peptide (SEQ ID NO: 26), leading to attenuating T cell exhaustion, thereby enhances the anti-tumor immune response both in the tumor microenvironment and systemically. e) Upregulate the anti-tumor immune response through blockage of PD-1 and PD-L1 interaction by anti-PD-L1 peptide (SEQ ID NO: 26); f) Immune cells infiltrate tumors, leading to converting a cold tumor microenvironment into a hot one and eliminating tumor.

FIG. 8. The Targeted Delivery and Mechanism of Action of the rCPB-conjugated Tandem Peptide-antioncogenic miRNA Chimeric Molecule. a) rCPB-conjugated tandem peptide-antioncogenic miRNA chimeric molecule binds to cancer cells through specific dual-binding of tandem peptide to cancer cells in which targeting peptide specifically binds to target cell and anti-PD-L1 peptide (SEQ ID NO: 26) specifically binds to PD-L1, while rCPB is removed from the chimeric molecule through cleavage of N-(-glutamyl) lysine isopeptide bond by TG highly expressed on cancer cells. Upon removing rCPB, tandem peptide-antioncogenic chimeric molecule is specifically delivered into cancer cells through synergistic role with the intracellular trafficking peptide, transportan (SEQ ID NO: 25), a component of the conjugate, in crossing cell membrane; b) Following the targeted delivery of the antioncogenic chimeric molecule into cancer cells, the double strands of antioncogenic miRNA dissociate, allowing the guide strand to be selected for loading into Argonaute, forming RISC, and then bind to target oncogenic miRNA while the first, second and third linkers are cleaved by cathepsin B which is highly expressed in cancer cells, leading to release of anti-PD-L1 peptide (SEQ ID NO: 26), passenger strand of antioncogenic miRNA and targeting peptide in cytoplasm; c) Anti-PD-L1 peptide (SEQ ID NO: 26) inhibit PD-L1; Within RISC, the guide strand binds to the Argonaute protein, preventing the oncogenic miRNA from binding to its target mRNA while the passenger strands are ejected and degraded; (d) Binding of antioncogenic miRNA to miRNA within RISC leads to blocking both translational repression and the target mRNA degradation, thereby induce cancer cell apoptosis and inhibits tumor growth through downregulation of oncogenic pathways by blocking both translational repression and the target mRNA degradation; PD-1 and PD-L1 interaction is blocked by anti-PD-L1 peptide (SEQ ID NO: 26), leading to attenuating T cell exhaustion, thereby enhances the anti-tumor immune response both in the tumor microenvironment and systemically. e) Upregulate the anti-tumor immune response via blockage of PD-1 and PD-L1 interaction by anti-PD-L1 peptide (SEQ ID NO: 26); f) Immune cells infiltrate tumors, leading to converting a cold tumor microenvironment into a hot one and eliminating tumor.

FIG. 9. The Targeted Delivery and Mechanism of Action of The rCPB-conjugated NoCltandem Peptide-miRNA Chimeric Molecule. a) rCPB-conjugated NoCltandem Peptide-miRNA Chimeric Molecule binds to cancer cells through specific binding of targeting peptide to target cells, while rCPB is removed from the chimeric molecule through cleavage of N-(-glutamyl) lysine isopeptide bond by TG highly expressed on cancer cells. Upon removing rCPB, the NoCltandem peptide-miRNA chimeric molecule is specifically delivered into cancer cells through synergistic role with the intracellular trafficking peptide, transportan (SEQ ID NO: 25), a component of the conjugate, in crossing cell membrane; b) Following the targeted delivery of the NoCltandem peptide-miRNA chimeric molecule into cancer cells, the double strands of miRNA dissociate, allowing the guide strand to be selected for loading into Argonaute, forming RISC, and then bind to target mRNA while the first and third linkers are cleaved by cathepsin B which is highly expressed in cancer cells, leading to release of passenger strand of miRNA and targeting peptide in cytoplasm; c) Binding of guide strand of miRNA to mRNA within RISC leads translational repression or the targeted mRNA degradation while the passenger strands are ejected and degraded; (d) Binding of miRNA to mRNA results in cancer cells apoptosis by translational repression or inducing the target mRNA degradation.

FIG. 10. The Targeted Delivery and Mechanism of Action of the rCPB-conjugated NoCltandem Peptide-antioncogenic miRNA Chimeric Molecule. a) rCPB-conjugated NoCltandem Peptide-antioncogenic miRNA Chimeric Molecule binds to cancer cells through specific binding of targeting peptide to target cells, while rCPB is removed from the chimeric molecule via cleavage of the N-(-glutamyl) lysine isopeptide bond by TG highly expressed on cancer cells. Upon removing rCPB, NoCltandem peptide-antioncogenic chimeric molecule is specifically delivered into cancer cells through synergistic role with the intracellular trafficking peptide, transportan (SEQ ID NO: 25), a component of the conjugate, in crossing cell membrane; b) Following the targeted delivery of the antioncogenic-miRNA chimeric molecule into cancer cells, the double strands of antioncogenic miRNA dissociate, allowing the guide strand to be selected for loading into Argonaute, forming RISC, and then bind to target oncogenic miRNA while first and third linkers are cleaved by cathepsin B which is highly expressed in cancer cells, leading to release of passenger strand of antioncogenic miRNA and targeting peptide in cytoplasm; c) Within RISC, the guide strand binds to the Argonaute protein, preventing the oncogenic miRNA from binding to its target mRNA while the passenger strands are ejected and degraded; (d) Binding of antioncogenic miRNA to miRNA within RISC leads to blocking both translational repression and the target mRNA degradation, thereby induce cancer cell apoptosis and inhibits tumor growth through downregulation of oncogenic pathways by blocking both translational repression and the target mRNA degradation.

FIG. 11. The Targeted Delivery and Mechanism of Action of rCPB-conjugated NoCltandem Peptide-DOX. a) rCPB-conjugated NoCltandem Peptide-DOX binds to cancer cells through specific binding of targeting peptide to target cells, while rCPB is removed from the conjugate through cleavage of N-(-glutamyl) lysine isopeptide bond by TG highly expressed on cancer cells. Upon removing rCPB, NoCltandem Peptide-Conjugated DOX is specifically delivered into cancer cells through synergistic role with the intracellular trafficking peptide, transportan (SEQ ID NO: 25), a component of the conjugate, in crossing cell membrane; b) Following the targeted delivery of the NoCltandem Peptide-DOX into cancer cells, the first and third linkers in the rCPB-conjugate are cleaved by cathepsin B which is highly expressed in cancer cells, leading to release of DOX and targeting peptide in cytoplasm; c) DOX damages DNA in nucleus, leading to cancer cell apoptosis; d) DOX induces immunogenic cell death, which promotes high DAMP signals for DC maturation and cytotoxic T lymphocyte activation, resulting in an increased anti-tumor immune response in the tumor microenvironment to eliminate tumor.

FIG. 12. The Targeted Delivery and Mechanism of Action of rCPB-conjugated NoCltandem Peptide-LLO. a) rCPB-conjugated NoCltandem Peptide-LLO binds to cancer cells through specific binding of targeting peptide to cancer cells, while rCPB is removed from the conjugate via cleavage of N-(-glutamyl) lysine isopeptide bond by TG highly expressed on cancer cells. Upon removing rCPB, NoCltandem Peptide-Conjugated LLO is specifically delivered into cancer cells through synergistic role with the intracellular trafficking peptide, transportan (SEQ ID NO: 25), a component of the conjugate, in crossing cell membrane; b) Following the targeted delivery of the NoCltandem Peptide-LLO into cancer cells, the first and third linkers in the conjugate are cleaved by cathepsin B which is highly expressed in cancer cells, leading to release of LLO and targeting peptide in cytoplasm; c) The LLO disrupts phagolysosomal membranes that enhance antigen cross-presentation of tumor-derived peptides and activate cytosolic immune sensors, and boost macrophage activation to a pro-inflammatory phenotype, leading to enhancing anti-tumor immune response in tumor microenvironment; d) LLO induces cancer cell death, resulting in an increased anti-tumor immune response in the tumor microenvironment to eliminate tumor.

### DETAILED DESCRIPTION OF THE INVENTION

The present invention retains the core components disclosed in the prior applications, application number, 19/017,738 and application Ser. No. 19/074,994. However, the present invention introduces significant advancements in reducing off-target toxicity for targeting cancers highly expressing TG by incorporating rCPB and the linker (CQGG) within a single molecule. I have specifically designed the rCPB and the linker (CQGG) to generate these innovative molecules. This approach enables the tandem peptide-cytotoxin conjugates, tandem peptide-RNA chimeric molecules, NoCltandem peptide-RNA chimeric molecules and NoCltandem peptide-cytotoxin can't be delivered into cancer cells without removing rCPB from the conjugates and the chimeric molecules through cleavage of N-(-glutamyl) lysine isopeptide bond by TG expressed on cancer cells. These innovative molecules are designed specifically for intervention in cancers which highly express TG. The innovative structure and functional design described herein provide a distinct invention for intervention in the cancer highly expressing TG.

Cancer remains a leading cause of morbidity and mortality worldwide, despite significant advancements in targeted therapies, immunotherapies, and antibody-drug conjugates (ADCs). Off-target toxicity presents a major barrier to effective cancer therapy. While targeted therapeutics aim to minimize damage to normal tissues, unintended interactions with non-malignant cells result in significant adverse effects. Antibody cross-reactivity, systemic toxicity of chemotherapeutics, and immune-related adverse events associated with checkpoint inhibitors all contribute to therapy limitations, reducing patient compliance and overall treatment success.

In addition to off-target toxicity, one of the greatest challenges in cancer treatment is overcoming therapeutic resistance, which arises due to downregulation of target, tumor heterogeneity, genetic mutations, and microenvironmental factors that enable cancer cells to evade therapy. Resistance mechanisms include target mutation and downregulation, drug efflux, metabolic alterations, and immune escape, ultimately leading to disease progression and treatment failure.

Another critical challenge is ensuring therapeutic efficacy across diverse tumor types. Tumor heterogeneity, limited drug penetration, and immune evasion mechanisms hinder the long-term success of both small-molecule drug and biologics. To enhance treatment outcomes, novel strategies such as combination therapy, tumor-specific activation mechanisms, and biomarker-driven patient selection are being explored.

My present innovation is dedicated to addressing key challenges in off-target toxicity, resistance development, and efficacy limitations in cancer therapy, with a focus on innovative strategies to enhance cancer cell-killing efficacy. By leveraging rational design and advanced conjugation technologies, the next generation of cancer therapeutics can achieve greater clinical success.

In my present invention, I have innovated eight types of molecules:
a) two types of rCPB-conjugated tandem peptide-Cytotoxin: rCPB-conjugated tandem peptide-DOX and rCPB-conjugated tandem peptide-LLO;
b) two types of rCPB-conjugated tandem peptide-RNA chimeric molecules: rCPB-conjugated tandem peptide-miRNA chimeric molecules and rCPB-conjugated tandem peptide-antioncogenic miRNA chimeric molecules;
c) two types of rCPB-conjugated NoCltandem peptide-RNA chimeric molecules: rCPB-conjugated NoCltandem peptide-miRNA chimeric molecules and rCPB-conjugated NoCltandem peptide-antioncogenic miRNA chimeric molecules;
d) two types of rCPB-conjugated NoCltandem peptide-Cytotoxin: rCPB-conjugated NoCltandem peptide-DOX and rCPB-conjugated NoCltandem peptide-LLO.
   Structure and Components of rCPB-Conjugated Tandem Peptide-Cytotoxin, rCPB-Conjugated Tandem Peptide-RNA Chimeric Molecules, rCPB-Conjugated NoCltandem Peptide-RNA Chimeric Molecules and rCPB-Conjugated NoCltandem Peptide-Cytotoxin

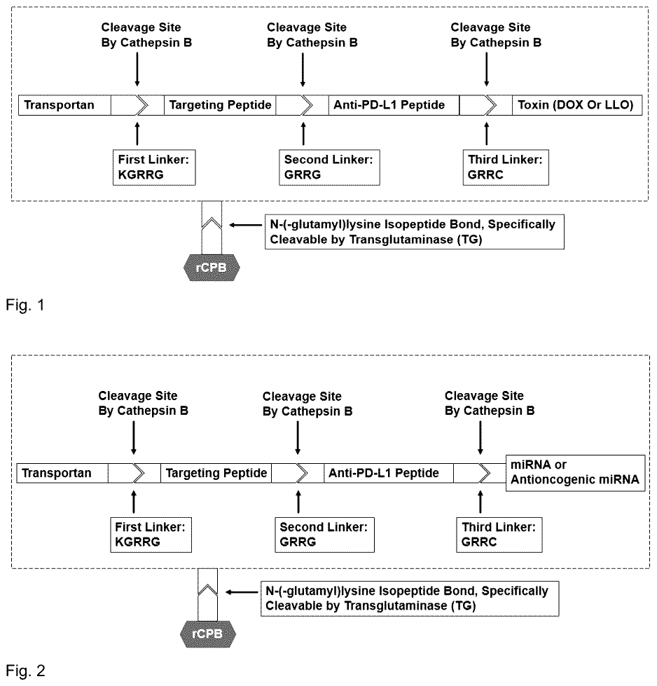

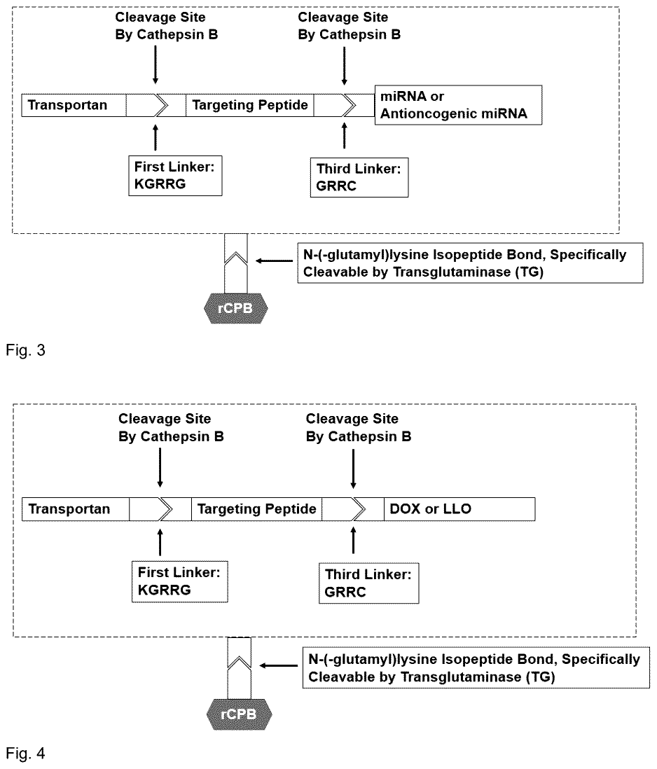

Tandem peptide for rCPB-conjugated tandem peptide-cytotoxin, rCPB-conjugated tandem peptide-miRNA chimeric molecules and rCPB-conjugated tandem peptide-antioncogenic miRNA chimeric molecules is same, which consists of transportan (GGWTLNSAGYLLGKINLKALAALAKKIL; SEQ ID NO: 25), first linker KGRRG (SEQ ID NO: 27), targeting peptide, second linker GRRG (SEQ ID NO: 28), anti-PD-L1 peptide (CVRARTR; SEQ ID NO: 26), and third linker GRRC (SEQ ID NO: 29) from N-terminus to C-terminus (FIG. 1-2). NoCltandem peptide is the same as tandem peptide, except for the absence of both second linker and anti-PD-L1 peptide (FIG. 3-4). Transportan (SEQ ID NO: 25) is an intracellular trafficking peptide. The targeting peptide is selected based on its binding specificity to different target cells (cancer). Tandem peptide contains the first, second and third linkers, while the NoCltandem peptide lacks the second linker. To maintain consistency, the linker names (first and third) are kept the same in the NoCltandem peptide, even though the second linker is absent.

The tandem peptide-cytotoxin is generated by conjugating tandem peptide to DOX or LLO through the third linker at C-terminus of tandem peptide.

Tandem peptide-RNA chimeric molecule (Tandem peptide-miRNA chimeric molecules and tandem peptide-antioncogenic miRNA chimeric molecules) is generated, comprising:
a) Thiolate passenger strand of miRNA or passenger strand of antioncogenic miRNA;
b) Hybridization of guide strand to the thiolated passenger strand of miRNA and thiolated passenger strand of antioncogenic miRNA, respectively, to generate double-stranded thiolated miRNA and thiolated antioncogenic miRNA;
c) Conjugate tandem peptide to 3′ end of the thiolated passenger strand of miRNA or 3′ end of thiolated passenger strand of antioncogenic miRNA through the third linker (GRRC) at C-terminus of the tandem peptide.

NoCltandem peptide-RNA chimeric molecule (NoCltandem peptide-miRNA chimeric molecules and NoCltandem peptide-antioncogenic miRNA chimeric) is generated in the same way as Tandem peptide-RNA chimeric molecule, with NoCltandem peptide replacing tandem peptide.

NoCltandem peptide-cytotoxin (NoCltandem peptide-DOX and NoCltandem peptide-LLO) is generated in the same way as Tandem peptide-cytotoxin, with NoCltandem peptide replacing tandem peptide.

rCPB is selected from the group of liposomes, NK-92 cells, cell membranes, and large molecules, that are involved in blocking the delivery of the conjugate and chimeric molecules into target cells. Fourth linker (CQGG) is conjugated to rCPB. rCPB-conjugated tandem peptide-cytotoxin, rCPB-conjugated tandem peptide-RNA chimeric molecule, rCPB-conjugated NoCltandem peptide-RNA chimeric molecule and rCPB-conjugated NoCltandem peptide-cytotoxin chimeric molecule are generated by conjugating rCPB to tandem peptide-cytotoxin, tandem peptide-RNA chimeric molecule, NoCltandem peptide-RNA chimeric molecules and NoCltandem peptide-cytotoxin, respectively, through fourth linker catalyzed by TG. The first, second and third linkers are designed for cleavable by cathepsin B which highly expresses in cancer cells compared to normal cells, leading to reducing off-get toxicity risk. Normal cells inherently exhibit low cathepsin B expression (5-8).

Transglutaminase not only catalyze N-(-glutamyl) lysine cross-links, connecting various peptide chain segments but also hydrolyze the N-(-glutamyl) lysine bond (γ:εlinkage) (1).

### Scientific Rational of Linker Design

In my present innovative molecules, I designed four distinct linkers: the first linker (SEQ ID NO: 27) is Lys-Gly-Arg-Arg-Gly (KGRRG), the second linker (SEQ ID NO: 28) is Gly-Arg-Arg-Gly (GRRG), the third linker (SEQ ID NO: 29) is Gly-Arg-Arg-Cys (GRRC), and the fourth linker (SEQ ID NO: 30) is Cys-Gln-Gly-Gly (CQGG). CQGG (SEQ ID NO: 30) is conjugated to rCPB by forming disulfide bond (—S—S—) between Cysteine (C) in CQGG and thiolated rCPB. CQGG is conjugated to tandem peptide or NoCltandem peptide by forming N-(-glutamyl) lysine isopeptide bond (γ:ε linkage) between glutamine (Q) in CQGG and lysin (K) in tandem peptide in presence of transglutaminase (TG), or between glutamine (Q) in CQGG and lysin (K) in NoCltandem peptide in presence of TG. The TG not only catalyze forming N-(-glutamyl) lysine isopeptide bond but also break the bond (1), thereby the rCPB can be removed through cleavage of N-(-glutamyl) lysine isopeptide bond by TG highly expressed on cancer cells. Furthermore, the active components are linked through the linkers. The first, second and third linkers are designed to possess Arg-Arg bond, thereby following targeted delivery of the rCPB-free conjugates or rCPB-free chimeric molecule into cancer cells, the active compounds are released upon cleavage of Arg-Arg (R—R) bond by cathepsin B highly expressed in cancer cells. Cathepsin B specifically cleaves Arg-Arg bond (9-11).

### Scientific and Functional Rationale of the Present Invention

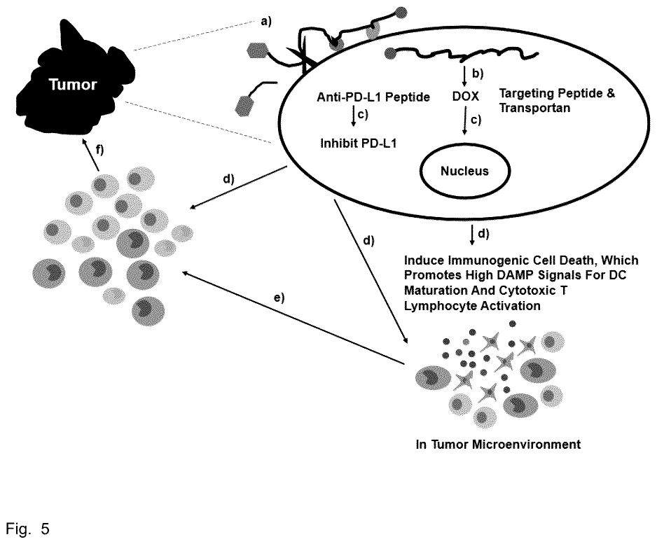

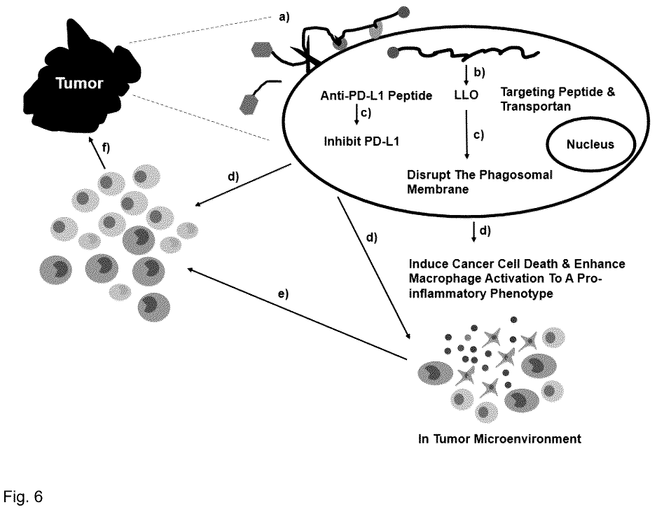

In my present innovations, the scientific rational (FIG. 5-6) behind these innovative molecules, rCPB-conjugated tandem peptide-DOX and rCPB-conjugated tandem peptide-LLO for intervention in cancers which highly express TG is that upon specifically binding of tandem peptide to cancer cells through specific dual-binding of the targeting peptide and anti-PD-L1 peptide (SEQ ID NO: 26) to their targets in cancer cells, the rCPB is removed from rCPB-conjugated tandem peptide-cytotoxin, through cleavage of N-(-glutamyl) lysine isopeptide bond by TG highly expressed on cancer cells. Following the rCPB removal, the tandem peptide-DOX or tandem peptide-LLO is delivered into cancer cells. The intracellular trafficking peptide, transportan (SEQ ID NO: 25), a component of the conjugate facilities the molecules in crossing cell membrane. Following targeted delivery of the tandem peptide-cytotoxin into cancer cells, anti-PD-L1 peptide, targeting peptide, DOX or LLO are released by cleaving the first, second and third linkers through cathepsin B. The anti-PD-L1 peptide attenuates T cell exhaustion to enhance anti-tumor immune response by inhibiting PD-L1. The released DOX induce immunogenic cell death (ICD) in cancer cells, which promotes high DAMP signals for dendritic cells (DC) maturation and cytotoxic T lymphocyte activation (12). The released LLO disrupts phagolysosomal membranes that enhance antigen cross-presentation of tumor-derived peptides and activate cytosolic immune sensors, and boost macrophage activation to a pro-inflammatory phenotype (13). Taken together, these conjugates kill cancer cells by targeting multiple pathways, including directly inducing cancer cell death, immune action and converting a cold tumor microenvironment into a hot one. Schrank et al. (2025) developed CD47-LLO. They reported that the release of LLO from CD47-LLO is critical to enhance macrophage activation to a pro-inflammatory phenotype (13).

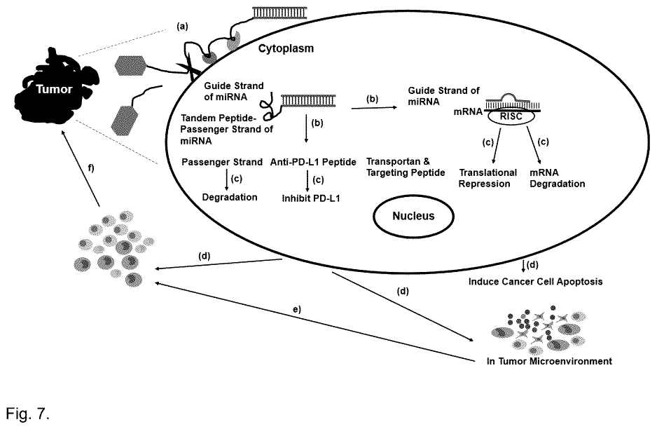

Scientific principle behind the rCPB-conjugated Tandem Peptide-miRNA Chimeric Molecule for the intervention in the cancers highly expressing TG (FIG. 7) is that rCPB-conjugated Tandem Peptide-miRNA Chimeric Molecule binds to cancer cells through specific dual-binding of tandem peptide to cancer cells in which targeting peptide specifically binds to target cell and anti-PD-L1 peptide (SEQ ID NO: 26) specifically binds to PD-L1, while rCPB is removed from the molecule through cleavage of N-(-glutamyl) lysine isopeptide bond by TG highly expressed on cancer cells. Upon removing rCPB, the tandem peptide-miRNA chimeric molecule is specifically delivered into cancer cells through synergistic role with the intracellular trafficking peptide, transportan (SEQ ID NO: 25), a component of the conjugate, in crossing cell membrane. Following the targeted delivery of the tandem peptide-miRNA chimeric molecule into cancer cells, the double strands of miRNA dissociate, allowing the guide strand to be selected for loading into Argonaute, forming RISC, and then bind to target mRNA while the first, second and third linkers are cleaved by cathepsin B which is highly expressed in cancer cells, leading to release of anti-PD-L1 peptide (SEQ ID NO: 26), passenger strand of miRNA and targeting peptide in cytoplasm. Binding of guide strand of miRNA to mRNA within RISC leads translational repression or the targeted mRNA degradation while the passenger strands are ejected and degraded. Binding of miRNA to mRNA results in cancer cells apoptosis by translational repression or inducing the target mRNA degradation. PD-1 and PD-L1 interaction is blocked by anti-PD-L1 peptide (SEQ ID NO: 26), leading to attenuating T cell exhaustion, thereby enhances the anti-tumor immune response both in the tumor microenvironment and systemically. Upregulation of the anti-tumor immune response via blockage of PD-1 and PD-L1 interaction by anti-PD-L1 peptide (SEQ ID NO: 26) results in immune cells infiltrate tumors, leading to converting a cold tumor microenvironment into a hot one and eliminating tumor.

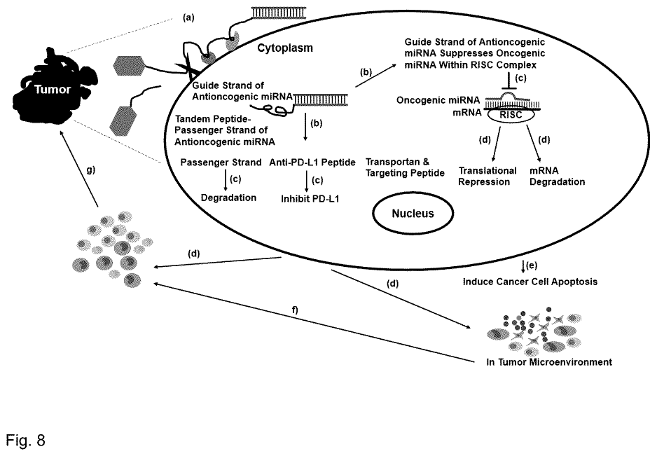

Scientific basis behind the rCPB-conjugated Tandem Peptide-antioncogenic miRNA Chimeric Molecule for the intervention in the cancers highly expressing TG (FIG. 8) is that rCPB-conjugated Tandem Peptide-antioncogenic miRNA Chimeric Molecule binds to cancer cells through specific dual-binding of tandem peptide to cancer cells in which targeting peptide specifically binds to target cell and anti-PD-L1 peptide (SEQ ID NO: 26) specifically binds to PD-L1, while rCPB is removed from the molecule via cleavage of the N-(-glutamyl) lysine isopeptide bond by TG highly expressed on cancer cells. Upon removing rCPB, the tandem peptide-antioncogenic miRNA chimeric molecule is specifically delivered into cancer cells through synergistic role with the intracellular trafficking peptide, transportan (SEQ ID NO: 25), a component of the conjugate, in crossing cell membrane. Following the targeted delivery of the tandem peptide-antioncogenic miRNA chimeric molecule into cancer cells, the double strands of antioncogenic miRNA dissociate, allowing the guide strand to be selected for loading into Argonaute, forming RISC, while the first, second and third linkers are cleaved by cathepsin B which is highly expressed in cancer cells, leading to release of anti-PD-L1 peptide (SEQ ID NO: 26), passenger strand of antioncogenic miRNA and targeting peptide in cytoplasm. Within RISC, the guide strand binds to the Argonaute protein, preventing the oncogenic miRNA from binding to its target mRNA while the passenger strands are ejected and degraded. Binding of antioncogenic miRNA to miRNA within RISC leads to blocking both translational repression and the target mRNA degradation, thereby induce cancer cell apoptosis and inhibits tumor growth through downregulation of oncogenic pathways by blocking both translational repression and the target mRNA degradation. PD-1 and PD-L1 interaction is blocked by anti-PD-L1 peptide (SEQ ID NO: 26), leading to attenuating T cell exhaustion, thereby enhances the anti-tumor immune response both in the tumor microenvironment and systemically. Upregulation of the anti-tumor immune response through blockage of PD-1 and PD-L1 interaction by anti-PD-L1 peptide (SEQ ID NO: 26) results in immune cells infiltrate tumors, leading to converting a cold tumor microenvironment into a hot one and eliminating tumor.

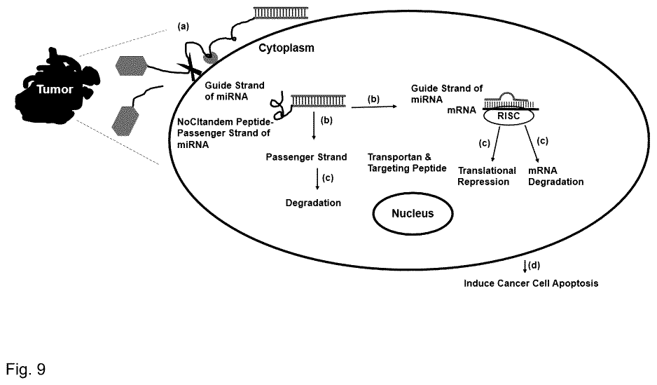

Scientific foundation behind the rCPB-conjugated NoCltandem Peptide-miRNA Chimeric Molecule for the intervention in the cancers highly expressing TG (FIG. 9) is that rCPB-conjugated NoCltandem Peptide-miRNA Chimeric Molecule binds to cancer cells through specific binding of targeting peptide to cancer cells, while rCPB is removed from the molecule through cleavage of N-(-glutamyl) lysine isopeptide bond by TG highly expressed on cancer cells. Upon removing rCPB, the NoCltandem peptide-miRNA chimeric molecule is specifically delivered into cancer cells through synergistic role with the intracellular trafficking peptide, transportan (SEQ ID NO: 25), a component of the conjugate, in crossing cell membrane. Following the targeted delivery of the NoCltandem peptide-miRNA chimeric molecule into cancer cells, the double strands of miRNA dissociate, allowing the guide strand to be selected for loading into Argonaute, forming RISC, and then bind to target mRNA while first and third linkers are cleaved by cathepsin B which is highly expressed in cancer cells, leading to release of passenger strand of miRNA and targeting peptide in cytoplasm. Binding of guide strand of miRNA to mRNA within RISC leads translational repression or the targeted mRNA degradation while the passenger strands are ejected and degraded. Binding of miRNA to mRNA results in cancer cells apoptosis by translational repression or inducing the target mRNA degradation.

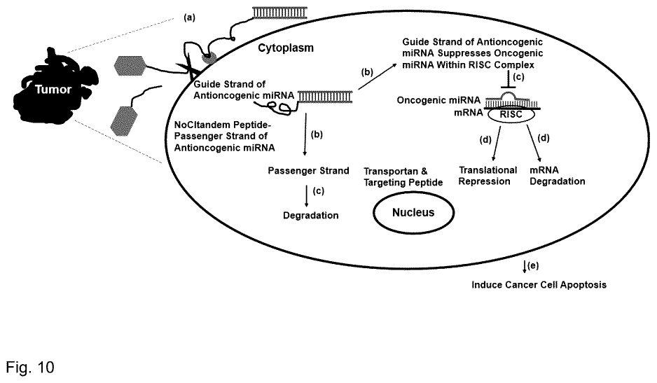

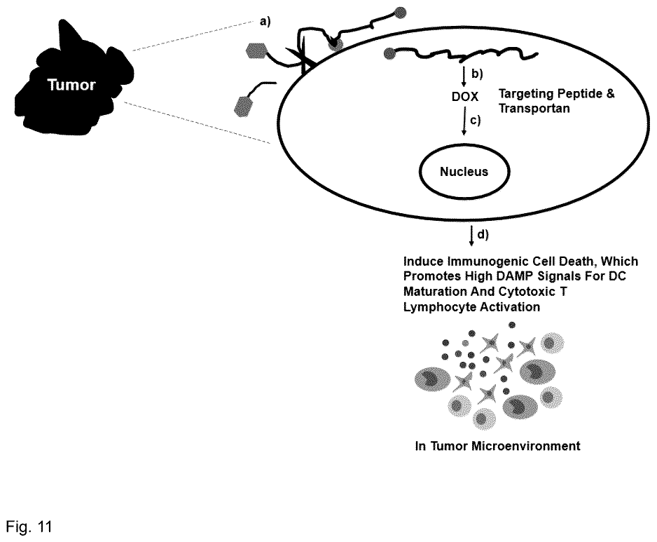

The underlying scientific mechanism behind the rCPB-conjugated NoCltandem Peptide-antioncogenic miRNA Chimeric Molecule for the intervention in the cancers highly expressing TG (FIG. 10) is that rCPB-conjugated NoCltandem Peptide-antioncogenic miRNA Chimeric Molecule binds to cancer cells through specifically binding of targeting peptide to cancer cell while rCPB is removed from the molecule through cleavage of N-(-glutamyl) lysine isopeptide bond by TG highly expressed on cancer cells. Upon removing rCPB, the tandem peptide-antioncogenic miRNA chimeric molecule is specifically delivered into cancer cells through synergistic role with the intracellular trafficking peptide, transportan (SEQ ID NO: 25), a component of the conjugate, in crossing cell membrane. Following the targeted delivery of the NoCltandem peptide-antioncogenic miRNA chimeric molecule into cancer cells, the double strands of antioncogenic miRNA dissociate, allowing the guide strand to be selected for loading into Argonaute, forming RISC, while first and third linkers are cleaved by cathepsin B which is highly expressed in cancer cells, leading to release of passenger strand of antioncogenic miRNA and targeting peptide in cytoplasm. Within RISC, the guide strand binds to the Argonaute protein, preventing the oncogenic miRNA from binding to its target mRNA while the passenger strands are ejected and degraded. Binding of antioncogenic miRNA to miRNA within RISC leads to blocking both translational repression and the target mRNA degradation, thereby induce cancer cell apoptosis and inhibits tumor growth through downregulation of oncogenic pathways by blocking both translational repression and the target mRNA degradation.

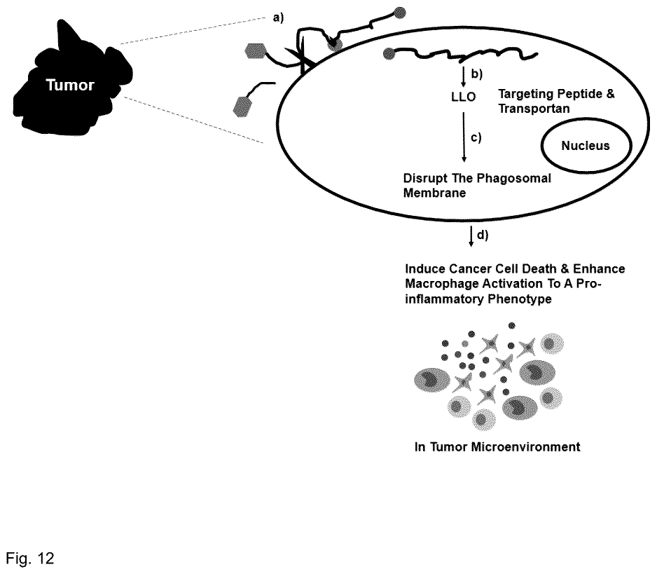

In my present innovations, the scientific rational (FIG. 11-12) behind these innovative molecules, rCPB-conjugated NoCltandem peptide-DOX and rCPB-conjugated NoCltandem peptide-LLO for intervention in cancers which highly express TG is that following specifically binding of targeting peptide to target cells, the rCPB is removed from rCPB-conjugated NoCltandem peptide-cytotoxin, through cleavage of N-(-glutamyl) lysine isopeptide bond by TG highly expressed on cancer cells. Upon the rCPB removal, the NoCltandem peptide-DOX or NoCltandem peptide-LLO is delivered into cancer cells. The intracellular trafficking peptide, transportan (SEQ ID NO: 25), a component of the conjugate facilities the molecules in crossing cell membrane. Following targeted delivery of the NoCltandem peptide-cytotoxin into cancer cells, targeting peptide, cytotoxin (DOX or LLO) is released by cleaving the first and third linkers through cathepsin B. The released DOX or LLO induce cancer cells death, as described above.

### Method

All tandem peptide and NoCltandem peptide are synthesized and acylated at N-terminus. The tandem peptide-conjugated LLO is generated following the method described by Schrank et al (2025) (13). Briefly, the tandem peptide is modified with the chemistry reagent DBCO-PEG4-NHS ester for 30 min at room temperature. Excess cross-linker was removed by high performance liquid chromatography (HPLC). The buffer of LLO protein is exchanged for PBS buffer, pH 7.2 with a PD-10 column (GE Healthcare, 17085101), followed by modification with the chemistry cross-linking reagent SPDP-PEG11-azide. Excess reagent was removed by buffer exchange, and then the modified LLO protein was mixed with tandem peptide and allowed to react overnight at room temperature. Unconjugated tandem peptide and LLO proteins were removed by affinity and size exclusion chromatographic methods, respectively. The generation of NoCltandem peptide-LLO is the same as tandem peptide-LLO, except that tandem peptide is replaced with NoCltandem peptide.

The tandem peptide-conjugated DOX is generated following the method described by Moon et al (12). In brief, the tandem peptide-conjugated DOX is synthesized by one-step conjugation of N-terminal acylated tandem peptide and doxorubicin (DOX) via EDC/NHS coupling chemistry. First, Ac-tandem peptide, DOX, NHS and EDC are dissolved in anhydrous DMF and then DIPEA is subsequently added in mixture solution. After 6 h of incubation, the compounds are purified using HPLC and lyophilized. The generation of NoCltandem peptide-DOX is the same as tandem peptide-DOX, except that tandem peptide is replaced with NoCltandem peptide.

Tandem peptide-RNA chimeric molecule (tandem peptide-miRNA chimeric molecules and tandem peptide-antioncogenic miRNA chimeric molecules) is generated by conjugating tandem peptide to passenger strand of miRNA or passenger strand of antioncogenic miRNA. In brief, 2′-O-methyl phosphoramidites and 2′-fluoro phosphoramidites are used to modify ribose of guide strand and passenger strand of RNAs (miRNA, and antioncogenic miRNA) at the 2′ position (14, 15). The passenger strand of miRNA or passenger strand of antioncogenic miRNA is synthesized with a terminal thiol group and then hybridize to their guide strand. The 3′ end of thiolated passenger strand of miRNA or 3′ end of thiolated passenger strand of antioncogenic miRNA is conjugated to tandem peptide through forming disulfide bond (—S—S—) between the third linker (GRRC) and (—SH) in the thiolated passenger strand.

NoCltandem peptide-RNA chimeric molecule (NoCltandem peptide-miRNA chimeric molecules and NoCltandem peptide-antioncogenic miRNA chimeric molecules) is generated in the same way as Tandem peptide-RNA chimeric molecule, with NoCltandem peptide replacing tandem peptide.

The fourth linker (CQGG) is synthesized. NK-92 cells, liposomes, cell membrane or large molecules is thiolated. The fourth linker (CQGG) is linked to the NK-92 cells, liposomes, cell membrane or large molecules via a thiol-reactive linkage. Following conjugating to the fourth linker (CQGG), the NK-92 cells, liposomes, cell membrane or large molecules is conjugated to tandem peptide-cytotoxin, tandem peptide-miRNA chimeric molecule, tandem peptide-antioncogenic miRNA chimeric molecule, NoCltandem peptide-cytotoxin, NoCltandem peptide-miRNA chimeric molecule, or NoCltandem peptide-antioncogenic miRNA chimeric molecule through forming N-(-glutamyl) lysine isopeptide bond between glutamine (Q) in fourth linker (CQGG) and lysine (K) in tandem peptide or between glutamine (Q) in fourth linker (CQGG) and lysine (K) in NoCltandem peptide in presence of TG (1). rCPB is conjugated to the tandem peptide or NoCltandem peptide, with potential to form additional conjugation with the LLO through forming N-(-glutamyl) lysine isopeptide bond between glutamine (Q) in fourth linker (CQGG) and lysine (K) in LLO in presence of TG; however, this does not affect the intended function of the conjugate because prior to targeted delivery into cancers cells, the rCPB is removed by TG highly expressed on cancer cells.

Experimental adjustments and optimizations may be necessary to achieve the desired product efficiently and selectively. The conjugate and chimeric molecule synthesis can be performed using alternative methods to those described above.

### Anti-PD-L1 Peptide (SEQ ID NO: 26), DOX and LLO Play Roles in Killing Cancer Cells

PD-L1 is expressed on several types of tumors, including urothelial cancers, gastrointestinal cancers, lung cancer, breast cancer, melanoma, and ovarian cancer, as well as on tumor infiltrating immune cells in the tumor microenvironment (16-22). Gong et al. reported PD-L1 expression in cell lines elevated after radiation via PI3K/AKT and STAT3 pathways. Moreover, down regulation of PD-L1 could reduce radiation resistance through promoting apoptosis. Radiotherapy in combination with anti-PD-L1 antibody synergistically elevated antitumor immunity through promoting CD8 T cell infiltration and decreasing the accumulation of MDSCs and tumor infiltrating regulatory T cells (iTregs) in a mouse model (23). The FDA approval for pembrolizumab which blocks binding of PD-L1 to PD-1 in lung cancer was received in October 2014 (24).

Tumor cells acquire different ways to evade the host immunity in the tumor microenvironment, called cancer immunosurveillance. Over the past decades, many studies of cancer immunosurveillance indicated that one of the superlative important components of the underlying mechanism is an immunosuppressive co-signal (immune checkpoint) mediated by PD-1/PD-L1 in the tumor microenvironment. Several preclinical reports also represented that blockage of the interaction between PD-1 and PD-L1 enhances the T-cell response and mediates antitumor activity (25-26). Anti-PD-1/PD-L1 therapy would make significant clinical benefits via promoting regression of advanced and metastatic tumors and improving survival. Importantly, anti-PD-1/PD-L1 therapy could have durable effects, tolerable toxicity, and is applicable to a wide spectrum type of cancers, especially in solid tumors (27, 26). Several PD-1 and PD-L1 inhibitors have been approved for cancer therapy by FDA (28).

DOX is most commonly used to treat cancers of the bladder, breast, stomach, lung, ovaries, thyroid, soft tissue sarcoma, multiple myeloma, and Hodgkin's lymphoma. The commonly used DOX-containing regimens may include Adriamycin, cyclophosphamide (AC), Taxotere, AC, Adriamycin, bleomycin, vinblastine, dacarbazine, bleomycin, etoposide, AC, vincristine, procarbazine, and prednisone, cyclophosphamide, Adriamycin, vincristine, prednisone and 5-fluorouracil, AC. Doxil is mainly used for the treatment of ovarian cancer where it has progressed or recurred after platinum-based chemotherapy, or for the treatment of AIDS-related Kaposi's sarcoma (29). The exact mechanism of action of DOX is complex and still unclear. DOX interacts with the DNA by intercalation thus, inhibiting macromolecular biosynthesis (30, 31). This further inhibits the progression of the enzyme topoisomerase II, and relaxes supercoils in DNA for transcription. DOX stabilizes the topoisomerase II complex after it has broken the DNA chain for replication, preventing the DNA double helix from being resealed and thereby stopping the process of replication. Another mechanism of DOX is its ability to generate free radicals that induce DNA and cell membrane damage (32).

LLO is a pH-dependent pore-forming cytotoxin with high cytolytic activity in acidic chambers. Bergelt et al. reported that LLO functions as the cytotoxic component of an immuno-cytotoxin, directly inducing tumor cell death (33). The B3-LLO immuno-cytotoxin has been strategically designed to exploit pH-dependent activation: in a neutral environment, LLO remains in an oxidized, low-cytotoxic state. However, upon internalization into acidic endosomal compartments, LLO regains maximal activity, disrupting the phagosomal membrane and inducing tumor cell death (33). This innovative approach establishes a novel platform for cancer immunotherapy.

Advancements in targeted liposome technology have enhanced the delivery of chemotherapeutic agents, enabling their direct accumulation within tumors. These drugs, capable of penetrating tumor cell membranes, improve treatment efficacy (34). However, systemic circulation inevitably exposes normal cells to cytotoxic effects, leading to off-target toxicity (35). Additionally, while some highly cytotoxic agents remain membrane-impermeable, their therapeutic potential is significantly higher when released into the cytosol (36, 37). LLO offers a promising solution to these challenges. Kullberg et al. developed an immunoliposome incorporating both LLO and bleomycin, a potent cytotoxic agent, for targeted delivery to HER2-overexpressing breast cancer cells. Trastuzumab facilitated tumor targeting, while LLO mediated liposomal cargo to pass into the cytoplasm, enabling cytosolic release of bleomycin (38). This approach led to a remarkable 57,000-fold increase in cytotoxicity compared to free bleomycin, underscoring LLO's potential in improving therapeutic outcomes (38).

The use of DOX and LLO as cytotoxins targeting cancer cells faces challenges related to toxicity and efficacy. To address this challenge, targeted delivery is imperative. In my present innovative conjugates, the DOX or LLO are conjugated to rCPB-conjugated tandem peptide. The conjugate can't be delivered into cancer cells without removing rCPB through cleavage of N-(-glutamyl) lysine isopeptide bond by TG expressed on cancer cells. Following the targeted delivery of rCPB-free conjugate into cancer cells, the linkers in conjugates are cleaved by cathepsin B to release active components. The components exert synergistic roles in killing cancer cells by directly inducing cancer cell apoptosis and enhancing anti-tumor immune response, converting a cold tumor microenvironment into a hot one.

Targeting Peptides' Roles in the Targeted Delivery of the Innovative Conjugate into Cancer Cells

Cell penetrating peptides are unique, 5-30 amino acid long peptides that are able to breach cell membrane barriers and carry cargoes intracellularly in a functional form. Sri et al. (2023) reported that peptides exhibit lower affinity and a shorter half-life in the body than antibodies. Conversely, peptides demonstrate higher efficiency in tissue penetration and cell internalization than antibodies. Regardless of the pros and cons of peptides, they have been used as tumor-homing ligands for delivering carriers (such as nanoparticles, extracellular vesicles, and cells) and cargoes (such as cytotoxic peptides and radioisotopes) to tumors. Additionally, tumor-homing peptides have been conjugated with cargoes such as small-molecule or chemotherapeutic drugs via linkers to synthesize peptide-drug conjugates. In addition, peptides selectively bind to cell surface receptors and proteins, such as immune checkpoints, receptor kinases, and hormone receptors, subsequently blocking their biological activity or serving as hormone analogs. Furthermore, peptides internalized into cells bind to intracellular proteins and interfere with protein-protein interactions. Thus, peptides demonstrate great application potential as multifunctional players in cancer therapy.

The peptides target cancers for the intervention in various cancers include RGD4C (ACDCRGDCFCG; SEQ ID NO: 1) targeting Integrin αvβ3 for the intervention in melanoma, colon tumor, ovarian tumor and glioblastoma (39-41), iRGD1 (CRGDR; SEQ ID NO: 2) and iRGD2 (KGPDC; SEQ ID NO: 3) targeting Integrin αvβ3 for the intervention in Glioblastoma and melanoma, LyP-1 (CGQKRTRGC; SEQ ID NO: 4) targeting P32 for the intervention in Melanoma (42), K237 (HTMYYHHYQHHL; SEQ ID NO: 5) targeting VEGFR-2 and mUNO (CSPGAK; SEQ ID NO: 6) targeting CD206 for the intervention in breast tumor (43, 44), Octreotide (FCFWKTCT; SEQ ID NO: 7) targeting SSTR2/5, specific for the intervention in pancreatic and breast tumor (45), IL4RPep-1 (CRKRLDRNC; SEQ ID NO: 8) targeting IL4R for the intervention in lung tumor, breast tumor and colon tumor (46-50), EPI-X4 (LVRYTKKVPQVSTPTL; SEQ ID NO: 9) targeting CXCR4 for the intervention in AML cells (51), TV1 (TRICCGCYWNGSKDVCSQSCC; SEQ ID NO: 10) targeting ion channels for the intervention in hepatocellular carcinoma (52), Bld-1 (CSNRDARRC; SEQ ID NO: 11) targeting bladder cancer cells for intervention in bladder cancer (53), AAD (AADNAKTKSFPV; SEQ ID NO: 12) and GP-5 (IHKDKNAPSLVP; SEQ ID NO: 13) targeting gastric cancer cell for intervention in gastric cancer (54, 55), LN1 (TGTPARQ; SEQ ID NO: 14), H3 (GFIATLCTKVLDFGIDKLQLIEDK; SEQ ID NO: 15) and Prost1 (WQPDTAHHWATL; SEQ ID NO: 16) targeting LNCaP prostate cancer cells for intervention in prostate cancer (56, 57), GE11 (YHWYGYTPQNVI; SEQ ID NO: 17) targeting EGFR for intervention in triple negative breast cancer, lung tumor, Laryngeal Cancer, head and neck tumor and squamous cell carcinoma of the skin (58-60), GIRLRG peptide (SEQ ID NO: 18) specifically bound to cervical, lung, esophageal, and glioma cells (61), hspb1 (RGPSWDPFRDWYPHSRLFDQAFGL; SEQ ID NO: 19) targeting Renal cell carcinoma (62), OSP-1 (ASGALSPSRLDT; SEQ ID NO: 20) targeting Osteosarcoma (63), nucleolin-binding F3 peptide (PEGASEMP; SEQ ID NO: 21) targeting Mesothelioma (64), RGD targeting Head and Neck Squamous Cell Carcinoma (HNSCC), thyroid cancer, lung cancer, Esophageal Cancer, Breast Cancer, Hepatocellular Carcinoma, Rectal Carcinoma, Renal Cancer, Prostate Cancer, Cervical Cancer, Ovarian Cancer and Osteosarcoma (65). BIRD2 (NVYTEIKCNSLLPLDDIVRV; SEQ ID NO: 22) binds to the BH4 domain, blocking Bcl-2-IP3 R interaction and thus inducing Ca2+-mediated apoptosis in chronic lymphocytic leukemia, multiple myeloma, and follicular lymphoma cells, including cells resistant to ABT-263, ABT-199, or the Bruton's tyrosine kinase inhibitor Ibrutinib (66, 67).

McCandless et al. (2021) identified a synthetic, non-naturally occurring 12-amino acid long peptide that was termed cardiac targeting peptide (CTP: APWHLSSQYSRT) due to its ability to transduce cardiomyocytes in vivo. Studies looking into its mechanism of transduction identified two lung targeting peptides (LTPs), S7A (APWHLSAQYSRT; SEQ ID NO: 23) and R11A (APWHLSSQYSAT; SEQ ID NO: 24) (68). These peptides robustly transduced human bronchial epithelial cell lines in vitro and mouse lung tissue in vivo. This uptake occurred independently of clathrin mediated endocytosis. Biodistribution studies of R11A showed peak uptake at 15 minutes with uptake in liver but not kidneys, indicating primarily a hepatobiliary mode of excretion. Cyclic version of both peptides was ~100-fold more efficient in permeating cells than their linear counterparts. As proof of principle, they conjugated anti-spike and anti-envelope SARS-COV-2 siRNAs to cyclized R11A and demonstrate anti-viral efficacy in vitro. Their work presented two novel lung-specific cell penetrating peptides that could potentially deliver myriad therapeutic cargoes to lung tissue (68).

In my present inventions, the targeting peptide, as a component of the innovative conjugates and chimeric molecules, plays roles in directed delivery of the conjugates and chimeric molecule into target cells.

miRNA's and Antioncogenic miRNA's Roles in Suppressing Cancers

miRNA, is small non-coding RNA, which has emerged as a revolutionary frontier in the field of medical science, offering groundbreaking possibilities in the treatment of multiple cancers.

Non-coding RNAs, much like transcription factors, have the ability to influence gene expression. Among them, miRNAs, discovered in 1993, stand out as small non-coding RNAs capable of modulating gene activity (69, 70). miRNA binds to mRNA, leading to repression of mRNA translation (71). miRNAs play a crucial role in diverse functions such as development, proliferation, differentiation, apoptosis, stress resistance, fat metabolism, survival, the tumorigenesis and metastasis of cells (72).

The post-transcriptional effect of miRNA gene expression, termed gene silencing, is primarily established through mRNA cleavage, translational repression, or DNA methylation (73, 74). RNase-like enzymes facilitate miRNA-mediated transcript cleavage, significantly impacting resulting mRNA expression (75). RNA helicase-regulated translational repression is essential for RNA degradation (76). The connection between DNA methylation and gene silencing, initially studied in plants, reveals that miRNAs regulate DNA methylation by targeting DNA methyltransferase enzymes (77). Such epigenetic modifications are now associated with tumor development (78, 79). While it was initially understood that miRNAs bind to the 3′ untranslated regions of their target genes (80-82), recent findings challenge this notion. Jopling et al. demonstrated that miR-122 in liver cells binds to the 5′ non-coding region of hepatitis C viral RNA. Additionally, Orom et al. confirmed that both 3′ and 5′ non-coding regions can act as miRNA binding sites (83).

Until recently, miRNAs were often dismissed as mere fragments of RNA. However, their significance has gained recognition with the discovery of aberrant expression in a diverse range of pathological events, particularly their involvement in carcinogenesis. This newfound attention has revealed their crucial roles as oncomiRs (oncogenic miRNA), where they inhibit tumor-suppressing genes, or as tumor suppressors, regulating oncogenes (84, 85).

Given that a single miRNA has the ability to target multiple genes, it possesses the capacity to influence numerous crucial pathways within the human body. Consequently, miRNAs play a pivotal role in the regulation of diverse biological functions such as metabolism, growth, development, immunity, and more (86). Their extensive range of targets underscores their capability to modulate the pathogenesis of cancers. miR-34 targeting pancreatic cancer (87), Let-7, anti-miR-181 and anti-miR10b targeting breast cancers (87), miR-34 and miR-126 targeting ovarian cancer (87), miR-101 and anti-miR-106b targeting prostate cancer (87), miR-203 targeting melanoma (88), miR-7 and miR-29 targeting lung cancer and colon cancer (87), anti-miR-155 targeting AML (86), miR-101 targeting bladder cancer (87), miR-199, miR-122, miR-148b, miR-195, anti-miR-21, anti-miR-93, anti-miR-221 and anti-miR-224 targeting Hepatocarcinoma cancer (89), miR-124 targeting Gastric Cancer, miR-34 targeting Renal cell carcinoma (87), miR-144, miR-100, miR-101, miR-125b, miR-133a, miR-138, miR-143, miR-145 and miR-424 targeting Cervical cancer (90), miR-181a-p, miR-144-p, miR-27b-p, miR-34a-p, miR-28-p, anti-miR-19b, anti-miR-21 and anti-miR-155-p targeting cell lymphoma (91), miR-23a and anti-miR-9 targeting Osteosarcoma (92, 93), miR-124 (94-96) and miR-126 (97-99) targeting Esophageal cancer, miR-34 and anti-miR-210 targeting renal cell carcinoma (87), miR-16-5p and miR-193a-3p targeting Mesothelioma (100, 101), and miR-125b-p, miR-145-p, miR-203, anti-miR-21-p, anti-miR-93-p, anti-miR-205-p and anti-miR-708 targeting Laryngeal Cancer (102).

### Rationale of not Obvious and Unexpected Advantages of the Invention

The present invention provides a novel class of conjugates and chimeric molecules for targeted intervention in cancers highly expressing TG, designed to overcome critical limitations of current therapeutic modalities. The molecular architecture disclosed herein offers not only enhanced efficacy and reduced intervention resistance through targeting multiple pathways, but also an unexpected and significant reduction in toxicity.

Unlike conventional antibody-drug conjugates (ADCs), other conjugates and chimeric molecules that often result in off-target toxicities due to premature release or non-specific uptake, the present invention employs a highly selective design strategy. In my innovative molecules for intervention in cancers highly expressing TG, I have specifically designed rCPB and fourth linker (CQGG) for conjugating rCPB to tandem peptide or to NoCltandem peptide by forming N(-glutamyl) lysine isopeptides (γ:ε linkage) between glutamine (Q) in fourth linker (CQGG) and lysine in tandem peptide or between glutamine (Q) in fourth linker (CQGG) and lysine in NoCltandem peptide. By leveraging this design, my present innovative molecules possess a three-layered mechanism to minimize off-target toxicity: (1) targeting peptide in NoCltandem peptide or both targeting peptide and anti-PD-L1 peptide (SEQ ID NO: 26) in tandem peptide directs conjugate or chimeric molecule to target cells by binding to their targets; (2) rCPB is removed through breaking N-(-glutamyl) lysine isopeptide bond by TG highly expressed on cancers cells to enable rCPB-free conjugate or rCPB-free chimeric molecule to deliver into cancer cells, thereby ensuring that normal cells are minimally affected, even within the same tissue; and (3) active components (anti-PD-L1, cytotoxin, miRNA, antioncogenic miRNA) is released through cleavage of specifically designed linkers by cathepsin B highly expressed in cancers to exert synergistic roles in immune response and gene regulation or directly kill cancer cells upon delivery into cells. Taken together, the innovative molecules achieve targeted delivery to cancer cells highly expressing TG, while sparing normal cells that coexist in the same tissue, thereby reducing off-target toxicity. These design elements act synergistically to confine the cytotoxic effect to malignant cells while sparing normal tissues.

This dual-functional outcome—simultaneous enhancement of efficacy and attenuation of toxicity—is a surprising and advantageous feature not anticipated in the prior art. In particular, the reduction of toxicity is not merely a theoretical benefit, but a direct consequence of the invention's structural and functional innovations. Such a reduction in off-target effects could be critical in expanding the therapeutic window and improving patient tolerability.

Given the inherent unpredictability in the design and function of biologic-drug conjugates or chimeric molecule, and the failure of prior art to suggest such combined improvements in therapeutic index, the present invention represents a non-obvious advancement in the field.

Although empirical efficacy and safety data are not included in this application, I provide herein a comprehensive scientific rationale that supports the innovative nature of this invention.

### Discussion

Cancer treatment faces significant challenges, including drug resistance, target downregulation, off-target toxicity, and limited efficacy. In my earlier innovative tandem peptide-cytotoxin (patent application Ser. No. 19/074,994), the strategy involves reducing off-target toxicity by combining the targeted delivery and incorporating linker which is specifically cleavable by cathepsin B that highly expresses in cancer cells for releasing anti-PD-L1 peptide (SEQ ID NO: 26), cytotoxin and targeting peptide, decreasing resistance by specifically dual-binding to target cells and targeting multiple pathways including directly killing cancer cells by cytotoxin, DOX or LLO, and anti-tumor immune action, and enhancing efficacy through leveraging synergistic roles in directly inducing cancer cells apoptosis by the cytotoxin and enhancing anti-tumor immune response via blockage of PD-1 and PD-L1 interaction by anti-PD-L1 peptide (SEQ ID NO: 26) and converting a cold tumor microenvironment into a hot one. In my earlier innovation (patent application Ser. No. 19/074,994) I designed two types of conjugates: tandem peptide-conjugated DOX and tandem peptide-conjugated LLO, each utilizing a distinct class of cytotoxic agent. Additionally, in my earlier innovative tandem peptide-miRNA chimeric molecules and tandem peptide-antioncogenic miRNA chimeric molecules (patent application Ser. No. 19/017,738), the strategy involves reducing off-target toxicity by targeted delivery of tandem peptide-miRNA chimeric molecules or tandem peptide-antioncogenic miRNA chimeric molecules into cancer cells through binding of targeting peptide to targets. However, in my present innovations, beyond that, I have specifically designed rCPB and a linker (CQGG) to develop rCPB-conjugated tandem peptide-cytotoxin, rCPB-conjugated tandem peptide-RNA chimeric molecules, rCPB-conjugated NoCltandem peptide-RNA chimeric molecules and rCPB-conjugated NoCltandem peptide-cytotoxin. The conjugates and chimeric molecules can't be delivered into cancer cells without removing rCPB from the conjugates and the chimeric molecules through cleavage of N-(-glutamyl) lysine isopeptide bond by TG expressed on cancer cells. This mechanism enables targeting of cancer cells without affecting normal cells coexisting in the same tissue, thereby significantly reducing toxicity. This approach significantly reduces off-target toxicity. TG is highly expressed in pancreatic cancer, breast cancer, ovarian cancer, epidermal squamous cell carcinoma, prostate cancer, glioma, melanoma, lung cancer, colon cancer, AML, bladder cancer, hepatocarcinoma, gastric cancer, renal cell carcinoma, cervical cancer, mantle cell lymphoma, osteosarcoma, esophageal cancer, mesothelioma and laryngeal cancer. This is distinct from my earlier patent applications, U.S. patent application Ser. No. 19/074,994 and U.S. patent application Ser. No. 19/017,738. Notably, this application does not claim priority to, nor is it a continuation of, either of those prior applications. My earlier patent applications, U.S. patent application Ser. No. 19/074,994 and U.S. patent application Ser. No. 19/017,738, cover a broader cancer spectrum. My present innovations are specifically directed toward the cancers which highly express TG.

Moon et al. (2022) developed anti-PD-L1 peptide-conjugated prodrug nanoparticles for targeted cancer immunotherapy. In their study, the functional peptide, consisted of anti-PD-L1 peptide (SEQ ID NO: 26) and cathepsin B-specific cleavable peptide, is conjugated to a DOX, resulting in prodrug nanoparticles of PD-NPs via intermolecular interactions. They reported that the PD-NPs are taken up by PD-L1 receptor-mediated endocytosis and then induce ICD in cancer cells by DOX release. Concurrently, PD-L1 blockade by PD-NPs disrupt the immune-suppressing pathway of cancer cells, resulting in proliferation and reinvigoration of T lymphocytes. In tumor models, PD-NPs accumulate within tumor tissues via enhanced permeability and retention (EPR) effect and induce immune-responsive tumors by recruiting a large number of immune cells (12). Importantly, my innovative conjugates are distinguishable from theirs. Beyond anti-PD-L1 peptide (SEQ ID NO: 26), linker and DOX, my innovative conjugates possess additional rCPB, fourth linker (CQGG) which is for forming N-(-glutamyl) lysine isopeptide bond between glutamine (Q) in CQGG and lysine (K) in tandem peptide or between glutamine (Q) in CQGG and lysine (K) in NoCltandem peptide, targeting peptide and an intracellular traffic peptide, transportan (SEQ ID NO: 25). The conjugate can't be delivered into cancer cells without removing rCPB from the conjugate through cleavage of N-(-glutamyl) lysine isopeptide bond by TG expressed on cancer cells, ensuring the specific targeting of cancer cells with minimal impact on normal cells, even in the presence of normal cells within the same tissue.

In my innovative rCPB-conjugated tandem peptide-cytotoxin, the targeting peptide and anti-PD-L1 peptide (SEQ ID NO: 26) play synergistic roles with intracellular traffic peptide in the targeted delivery of the conjugates into cancer cell. Tumor cells frequently develop resistance through mechanisms such as downregulation of receptor and targets, activation of alternative survival pathways and genetic mutations to escape attacking. In my innovative conjugates, both targeting peptide and anti-PD-L1 peptide (SEQ ID NO: 26) bind to target cells. This dual-binding strategy reduces the risk of escape mechanisms, leading to decreasing resistance development and a lower likelihood of cancer relapse.

Schrank et al (2025) developed a CD47 antibody-conjugated LLO for cancer immunotherapy. Using a microbial-inspired strategy, they engineered an antibody-cytotoxin conjugate (ATC) that targets the ‘don't eat me’ signal CD47 linked to the bacterial cytotoxin, LLO, from the intracellular bacterium Listeria monocytogenes via a cleavable linker (CD47-LLO). CD47-LLO promotes cancer cell phagocytosis by macrophages followed by LLO release and activation to form pores on phagolysosomal membranes that enhance antigen cross-presentation of tumor-derived peptides and activate cytosolic immune sensors. CD47-LLO treatment in vivo significantly inhibited the growth of both localized and metastatic breast and melanoma tumors and improved animal survival as a monotherapy or in combination with checkpoint blockade. Together, their results demonstrate that designing ATCs to promote immune recognition of tumor cells represents a promising therapeutic strategy for treating multiple cancer (13). Their conjugates utilize an antibody (Ab) as a binding ligand to specifically target cancer-associated antigens. However, in my present innovative conjugates, targeting peptide is as a binding ligand to bind cancer cells. Beyond the targeting peptide, my present innovative conjugates possess additional rCPB, fourth linker (CQGG) which is for formation of N-(-glutamyl) lysine isopeptide bond between glutamine (Q) in CQGG and lysine (K) in tandem peptide or between glutamine (Q) in CQGG and lysine (K) in NoCltandem peptide, targeting peptide and an intracellular traffic peptide, transportan (SEQ ID NO: 25), and anti-PD-L1 peptide (SEQ ID NO: 26). The rCPB and fourth linker (CQGG) are designed to ensure for specifically intervention in cancer cells highly expressing TG. The role of rCPB and fourth linker (CQGG) are described above.

By leveraging dual-binding of targeting peptide and anti-PD-L1 peptide (SEQ ID NO: 26) to target cells, this dual-binding strategy reduces the risk of escape mechanisms, leading to decreasing resistance development and a lower likelihood of cancer relapse. Anti-PD-L1 peptide (SEQ ID NO: 26) not only plays roles in specifically binding to target cells but also enhance anti-tumor immune response and promoting converting a cold tumor microenvironment into a hot one by inhibiting PD-L1 to attenuate T cell exhaustion.

Notably, although individual components or mechanisms described herein may be supported by prior literature, my present invention is based on a novel and non-obvious integration of these components in a specific and synergistic manner that yields functional advantages not suggested or taught in the prior art. The invention described herein is not a mere aggregation of known components, but rather a purposeful integration of structural and functional elements designed to achieve a synergistic effect and a novel molecular construct with properties that reduce toxicity risk, enhance efficacy, and decrease resistance.

Importantly, in my present innovative conjugate and chimeric molecules, I designed rCPB and fourth linker (CQGG) which is for formation of N-(-glutamyl) lysine isopeptide bond between glutamine (Q) in CQGG and lysine (K) in tandem peptide or between glutamine (Q) in CQGG and lysine (K) in NoCltandem peptide. The conjugate and chimeric molecules can't be delivered into cancers without removing rCPB from the conjugate and chimeric molecule through cleavage of N-(-glutamyl) lysine isopeptide bond by TG highly expressed on cancer cells, ensuing specific targeting of the cancer cells highly expressing TG without affecting coexisting normal cells, even within the same tissue. The present strategy represents a novel and non-obvious approach, with an associated mechanism that is significantly distinct from any prior art.

### Rationale Behind the Design of Various Types of Conjugates and Chimeric Molecules for Intervention in Cancers Highly Expressing TG

The development of multiple types of conjugates and chimeric molecules in this invention is a strategic approach to address the complexity and heterogeneity of cancer biology. The development of two distinct types of chimeric molecules-one containing an anti-PD-L1 and one without-provides a strategic and flexible approach to cancer therapy. The inclusion of a checkpoint inhibitor in one chimeric is intended to enhance anti-tumor r immune responses by reversing tumor-induced immunosuppression, thereby potentiating the therapeutic efficacy in immunologically active tumors or in settings where immune evasion is a key driver of disease progression.

Conversely, the chimeric molecules without the checkpoint inhibitor offers advantages in clinical scenarios where immune checkpoint blockade may be unnecessary, ineffective, or associated with increased risk of immune-related adverse events. This version provides a targeted cytotoxic or intervention effect without immune modulation, which may be preferable in certain tumor types or patient populations with limited immune responsiveness or contraindications to checkpoint inhibitors.

By developing both versions, I aim to address the heterogeneity of cancer biology, enabling tailored treatment strategies, broader clinical applicability, and the potential for combination or sequential therapy approaches to overcome resistance mechanisms and optimize efficacy.

Cancer cells often evade therapy through various mechanisms. Even though my innovative molecules incorporate strategies to reduce resistance development, the design of innovative molecules targeting different pathways increases the likelihood that at least one remains effective, even if resistance arises against the other.

The inclusion of diverse conjugate and chimeric formats ensures broader applicability, improved efficacy, and adaptability to current and future clinical needs.

### IN CONCLUSION

The rCPB-conjugated tandem peptide-cytotoxin, rCPB-conjugated tandem peptide-miRNA chimeric molecule, rCPB-conjugated tandem peptide-antioncogenic miRNA chimeric molecule, rCPB-conjugated NoCltandem peptide-miRNA chimeric molecule, rCPB-conjugated NoCltandem peptide-antioncogenic miRNA chimeric molecule and rCPB-conjugated NoCltandem peptide-cytotoxin were invented for intervention in various malignancies. Importantly, in my present innovations, rCPB and fourth linker (CRGG) are designed to develop these molecules. The conjugate or chimeric molecules can't be delivered into cancer cells without removing rCPB from the conjugates and the chimeric molecules through cleavage of N-(-glutamyl) lysine isopeptide bond by TG expressed on cancer cells. This approach significantly reduces off-target toxicity by specifically targeting cancer cells, sparing normal cells that coexist within the same tissue, making it a promising strategy for cancer therapy.

1. A rCPB-conjugated tandem peptide-RNA Chimeric Molecule, configured for intervention in lung cancer, Colon cancer or both, wherein cancer is characterized by high expression of members of transglutaminase (TG) family, comprising:
1) wherein rCPB is NK-92;
2) wherein rCPB is conjugated to tandem peptide through forming N-(-glutamyl) lysine isopeptide bond between glutamine (Q) in fourth linker (SEQ ID NO: 30) which is conjugated to rCPB and lysine (K) in tandem peptide catalyzed by transglutaminase (TG);
3) wherein the tandem peptide comprises a transportan (SEQ ID NO: 25), a first linker (SEQ ID NO: 27), a targeting peptide, a second linker (SEQ ID NO: 28), an anti-PD-L1 peptide (SEQ ID NO: 26), and a third linker (SEQ ID NO: 29) from N-terminus to C-terminus;
4) wherein the double-stranded RNA is miRNA;
5) wherein the tandem peptide is conjugated to 3′ end of passenger strand of miRNA through third linker (SEQ ID NO: 29) at C-terminus of the tandem peptide;
6) wherein first linker (SEQ ID NO: 27), second linker (SEQ ID NO. 28) and third linker (SEQ ID NO: 29) are peptides, specifically cleavable by cathepsin B expressed in cancer cells;
7) wherein N-(-glutamyl) lysine isopeptide bond is cleavable by TG expressed on cancer cells;
wherein the targeting peptide is IL4RPep-1 (SEQ ID NO: 8); and wherein IL4RPep-1 (SEQ ID NO: 8) specifically binds to a target expressed on the lung and colon cancer cells;
wherein the miRNA is miR-29 that induces death of lung cancer cells, colon cancer cells, or both;
wherein 2′-O-methyl phosphoramidites and 2′-fluoro phosphoramidites modify ribose of guide strand and passenger strand of miRNA at the 2′ position, thereby reducing degradation of the miRNA; and
wherein the tandem peptide are N-terminally acylated.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)


SEQUENCE LISTING (70 sequences)

SEQ ID NO: 1 - protein, 11 aa, synthetic construct
  PEPTIDE 1..11: note = RGD4C
ACDCRGDCFCG

SEQ ID NO: 2 - protein, 5 aa, synthetic construct
  PEPTIDE 1..5: note = iRGD1
CRGDR

SEQ ID NO: 3 - protein, 5 aa, synthetic construct
  PEPTIDE 1..5: note = iRGD2
KGPDC

SEQ ID NO: 4 - protein, 9 aa, synthetic construct
  PEPTIDE 1..9: note = LyP-1
CGQKRTRGC

SEQ ID NO: 5 - protein, 12 aa, synthetic construct
  PEPTIDE 1..12: note = K237
HTMYYHHYQHHL

SEQ ID NO: 6 - protein, 6 aa, synthetic construct
  PEPTIDE 1..6: note = mUNO
CSPGAK

SEQ ID NO: 7 - protein, 8 aa, synthetic construct
  PEPTIDE 1..8: note = Octreotide
FCFWKTCT

SEQ ID NO: 8 - protein, 9 aa, synthetic construct
  PEPTIDE 1..9: note = IL4RPep-1
CRKRLDRNC

SEQ ID NO: 9 - protein, 16 aa, synthetic construct
  PEPTIDE 1..16: note = EPI-X4
LVRYTKKVPQVSTPTL

SEQ ID NO: 10 - protein, 21 aa, synthetic construct
  PEPTIDE 1..21: note = TV1
TRICCGCYWNGSKDVCSQSCC

SEQ ID NO: 11 - protein, 9 aa, synthetic construct
  PEPTIDE 1..9: note = Bld-1
CSNRDARRC

SEQ ID NO: 12 - protein, 12 aa, synthetic construct
  PEPTIDE 1..12: note = AAD
AADNAKTKSFPV

SEQ ID NO: 13 - protein, 12 aa, synthetic construct
  PEPTIDE 1..12: note = GP-5
IHKDKNAPSLVP

SEQ ID NO: 14 - protein, 7 aa, synthetic construct
  PEPTIDE 1..7: note = LN1
TGTPARQ

SEQ ID NO: 15 - protein, 24 aa, synthetic construct
  PEPTIDE 1..24: note = H3
GFIATLCTKVLDFGIDKLQLIEDK

SEQ ID NO: 16 - protein, 12 aa, synthetic construct
  PEPTIDE 1..12: note = Prost1
WQPDTAHHWATL

SEQ ID NO: 17 - protein, 12 aa, synthetic construct
  PEPTIDE 1..12: note = GE11
YHWYGYTPQNVI

SEQ ID NO: 18 - protein, 6 aa, synthetic construct
  PEPTIDE 1..6: note = GIRLRG peptide
GIRLRG

SEQ ID NO: 19 - protein, 24 aa, synthetic construct
  PEPTIDE 1..24: note = hspb1
RGPSWDPFRDWYPHSRLFDQAFGL

SEQ ID NO: 20 - protein, 12 aa, synthetic construct
  PEPTIDE 1..12: note = OSP-1
ASGALSPSRLDT

SEQ ID NO: 21 - protein, 8 aa, synthetic construct
  PEPTIDE 1..8: note = nucleolin-binding F3 peptide
PEGASEMP

SEQ ID NO: 22 - protein, 20 aa, synthetic construct
  PEPTIDE 1..20: note = BIRD2
NVYTEIKCNSLLPLDDIVRV

SEQ ID NO: 23 - protein, 12 aa, synthetic construct
  PEPTIDE 1..12: note = S7A
APWHLSAQYSRT

SEQ ID NO: 24 - protein, 12 aa, synthetic construct
  PEPTIDE 1..12: note = R11A
APWHLSSQYSAT

SEQ ID NO: 25 - protein, 28 aa, synthetic construct
  PEPTIDE 1..28: note = Transportan
GGWTLNSAGYLLGKINLKALAALAKKIL

SEQ ID NO: 26 - protein, 7 aa, synthetic construct
  PEPTIDE 1..7: note = Anti-PD-L1 Peptide
CVRARTR

SEQ ID NO: 27 - protein, 5 aa, synthetic construct
  PEPTIDE 1..5: note = First linker
KGRRG

SEQ ID NO: 28 - protein, 4 aa, synthetic construct
  PEPTIDE 1..4: note = Second linker
GRRG

SEQ ID NO: 29 - protein, 4 aa, synthetic construct
  PEPTIDE 1..4: note = Third linker
GRRC

SEQ ID NO: 30 - protein, 4 aa, synthetic construct
  PEPTIDE 1..4: note = Fourth linker
CQGG

SEQ ID NO: 31 - RNA, 22 nt, Homo sapiens
  source 1..22: mol_type = other RNA
  gene 1..22: gene = hsa-miR-34a-5p
tggcagtgtcttagctggttgt

SEQ ID NO: 32 - RNA, 22 nt, Homo sapiens
  source 1..22: mol_type = other RNA
  gene 1..22: gene = hsa-let-7a-5p
tgaggtagtaggttgtatagtt

SEQ ID NO: 33 - RNA, 22 nt, Homo sapiens
  source 1..22: mol_type = other RNA
  gene 1..22: gene = hsa-miR-126-3p
tcgtaccgtgagtaataatgcg

SEQ ID NO: 34 - RNA, 21 nt, Homo sapiens
  source 1..21: mol_type = other RNA
  gene 1..21: gene = hsa-miR-101-3p
tacagtactgtgataactgaa

SEQ ID NO: 35 - RNA, 22 nt, Homo sapiens
  source 1..22: mol_type = other RNA
  gene 1..22: gene = hsa-miR-203a-3p
gtgaaatgtttaggaccactag

SEQ ID NO: 36 - RNA, 24 nt, Homo sapiens
  source 1..24: mol_type = other RNA
  gene 1..24: gene = hsa-miR-7-5p
tggaagactagtgattttgttgtt

SEQ ID NO: 37 - RNA, 22 nt, Homo sapiens
  source 1..22: mol_type = other RNA
  gene 1..22: gene = hsa-miR-29a-3p
tagcaccatctgaaatcggtta

SEQ ID NO: 38 - RNA, 22 nt, Homo sapiens
  source 1..22: mol_type = other RNA
  gene 1..22: gene = hsa-miR-199a-3p
acagtagtctgcacattggtta

SEQ ID NO: 39 - RNA, 22 nt, Homo sapiens
  source 1..22: mol_type = other RNA
  gene 1..22: gene = hsa-miR-122-5p
tggagtgtgacaatggtgtttg

SEQ ID NO: 40 - RNA, 22 nt, Homo sapiens
  source 1..22: mol_type = other RNA
  gene 1..22: gene = hsa-miR-148b-5p
aagttctgttatacactcaggc

SEQ ID NO: 41 - RNA, 21 nt, Homo sapiens
  source 1..21: mol_type = other RNA
  gene 1..21: gene = hsa-miR-195-5p
tagcagcacagaaatattggc

SEQ ID NO: 42 - RNA, 22 nt, Homo sapiens
  source 1..22: mol_type = other RNA
  gene 1..22: gene = hsa-miR-124-3p
taaggcacgcggtgaatgccaa

SEQ ID NO: 43 - RNA, 22 nt, Homo sapiens
  source 1..22: mol_type = other RNA
  gene 1..22: gene = hsa-miR-144-5p
ggatatcatcatatactgtaag

SEQ ID NO: 44 - RNA, 22 nt, Homo sapiens
  source 1..22: mol_type = other RNA
  gene 1..22: gene = hsa-miR-100-5p
aacccgtagatccgaacttgtg

SEQ ID NO: 45 - RNA, 22 nt, Homo sapiens
  source 1..22: mol_type = other RNA
  gene 1..22: gene = hsa-miR-125b-5p
tccctgagaccctaacttgtga

SEQ ID NO: 46 - RNA, 22 nt, Homo sapiens
  source 1..22: mol_type = other RNA
  gene 1..22: gene = hsa-miR-133a-3p
tttggtccccttcaaccagctg

SEQ ID NO: 47 - RNA, 23 nt, Homo sapiens
  source 1..23: mol_type = other RNA
  gene 1..23: gene = hsa-miR-138-5p
agctggtgttgtgaatcaggccg

SEQ ID NO: 48 - RNA, 21 nt, Homo sapiens
  source 1..21: mol_type = other RNA
  gene 1..21: gene = hsa-miR-143-3p
tgagatgaagcactgtagctc

SEQ ID NO: 49 - RNA, 23 nt, Homo sapiens
  source 1..23: mol_type = other RNA
  gene 1..23: gene = hsa-miR-145-5p
gtccagttttcccaggaatccct

SEQ ID NO: 50 - RNA, 22 nt, Homo sapiens
  source 1..22: mol_type = other RNA
  gene 1..22: gene = hsa-miR-424-5p
cagcagcaattcatgttttgaa

SEQ ID NO: 51 - RNA, 23 nt, Homo sapiens
  source 1..23: mol_type = other RNA
  gene 1..23: gene = hsa-miR-181a-5p
aacattcaacgctgtcggtgagt

SEQ ID NO: 52 - RNA, 21 nt, Homo sapiens
  source 1..21: mol_type = other RNA
  gene 1..21: gene = hsa-miR-27b-3p
ttcacagtggctaagttctgc

SEQ ID NO: 53 - RNA, 22 nt, Homo sapiens
  source 1..22: mol_type = other RNA
  gene 1..22: gene = hsa-miR-28-5p
aaggagctcacagtctattgag

SEQ ID NO: 54 - RNA, 21 nt, Homo sapiens
  source 1..21: mol_type = other RNA
  gene 1..21: gene = hsa-miR-23a-3p
atcacattgccagggatttcc

SEQ ID NO: 55 - RNA, 22 nt, Homo sapiens
  source 1..22: mol_type = other RNA
  gene 1..22: gene = hsa-miR-16-5p
tagcagcacgtaaatattggcg

SEQ ID NO: 56 - RNA, 22 nt, Homo sapiens
  source 1..22: mol_type = other RNA
  gene 1..22: gene = hsa-miR-193a-3p
aactggcctacaaagtcccagt

SEQ ID NO: 57 - RNA, 23 nt, synthetic construct
  source 1..23: mol_type = other RNA
  gene 1..23: gene = anti-hsa-miR-181a-5p
actcaccgacagcgttgaatgtt

SEQ ID NO: 58 - RNA, 23 nt, synthetic construct
  source 1..23: mol_type = other RNA
  gene 1..23: gene = anti-hsa-miR-10b-5p
cacaaattcggttctacagggta

SEQ ID NO: 59 - RNA, 20 nt, synthetic construct
  source 1..20: mol_type = other RNA
  gene 1..20: gene = anti-hsa-miR-106b-5p
atctgcactgtcacacttta

SEQ ID NO: 60 - RNA, 24 nt, synthetic construct
  source 1..24: mol_type = other RNA
  gene 1..24: gene = anti-hsa-miR-155-5p
aacccctatcacgattagcattaa

SEQ ID NO: 61 - RNA, 22 nt, synthetic construct
  source 1..22: mol_type = other RNA
  gene 1..22: gene = anti-hsa-miR-21-5p
tcaacatcagtctgataagcta

SEQ ID NO: 62 - RNA, 23 nt, synthetic construct
  source 1..23: mol_type = other RNA
  gene 1..23: gene = anti-hsa-miR-93-5p
ctacctgcacgaacagcactttg

SEQ ID NO: 63 - RNA, 22 nt, synthetic construct
  source 1..22: mol_type = other RNA
  gene 1..22: gene = anti-hsa-miR-221-5p
aaatctacattgtatgccaggt

SEQ ID NO: 64 - RNA, 25 nt, synthetic construct
  source 1..25: mol_type = other RNA
  gene 1..25: gene = anti-hsa-miR-224-5p
ctaaacggaaccactagtgacttga

SEQ ID NO: 65 - RNA, 23 nt, synthetic construct
  source 1..23: mol_type = other RNA
  gene 1..23: gene = anti-hsa-miR-19b-1-5p
gctggatgcaaacctgcaaaact

SEQ ID NO: 66 - RNA, 23 nt, synthetic construct
  source 1..23: mol_type = other RNA
  gene 1..23: gene = anti-hsa-miR-9-5p
tcatacagctagataaccaaaga

SEQ ID NO: 67 - RNA, 22 nt, synthetic construct
  source 1..22: mol_type = other RNA
  gene 1..22: gene = anti-hsa-miR-210-3p
tcagccgctgtcacacgcacag

SEQ ID NO: 68 - RNA, 23 nt, synthetic construct
  source 1..23: mol_type = other RNA
  gene 1..23: gene = anti-hsa-miR-93-5p
ctacctgcacgaacagcactttg

SEQ ID NO: 69 - RNA, 22 nt, synthetic construct
  source 1..22: mol_type = other RNA
  gene 1..22: gene = anti-hsa-miR-205-5p
cagactccggtggaatgaagga

SEQ ID NO: 70 - RNA, 23 nt, synthetic construct
  source 1..23: mol_type = other RNA
  gene 1..23: gene = anti-hsa-miR-708-5p
cccagctagattgtaagctcctt